(19) United States
(12) Patent Application Publication
(10) Pub. No.: US 2026/0266762 A1
(43) Pub. Date: Sep. 10, 2026

(54) DROPLET-ELECTROPHORESIS DEVICES AND RELATED METHODS

(71) Applicants: THE REGENTS OF THE UNIVERSITY OF CALIFORNIA, Oakland, CA (US); CZ BIOHUB SF, LLC, San Francisco, CA (US)

(72) Inventors: Amy E. Herr, Berkeley, CA (US); Yang Liu, Beijing (CN)

(21) Appl. No.: 19/473,399

(22) PCT Filed: Apr. 11, 2024
(86) PCT No.: PCT/US2024/024096

§ 371 Date: Oct. 7, 2025

Related U.S. Application Data
(60) Provisional application No. 63/458,866, filed on Apr. 12, 2023.

(51) Int. Cl. G01N 27/447; B01L 3/00; G01N 33/50

(52) U.S. Cl. CPC G01N 27/44782; B01L 3/502715; G01N 27/44743; G01N 27/44747; G01N 33/5005; B01L 2300/0829; B01L 2300/0864; B01L 2400/02

(57) ABSTRACT

Droplet-electrophoresis devices and related methods are disclosed. In accordance with an implementation, an apparatus includes a droplet generator, a housing, a microwell assembly, and a droplet loading device. The housing includes a perimeter wall including an interior side wall and a lip that define a receptacle. The microwell assembly is positioned within the receptacle and rests on the lip. The microwell assembly includes a plurality of microwells. The droplet loading device is positioned within the receptacle on top of the microwell assembly. The droplet loading device includes an inlet manifold assembly including an inlet, an outlet manifold assembly including an outlet, and a plurality of channels fluidly coupled to the inlet manifold assembly and the outlet manifold assembly. Each of the channels have an opening positioned over top of corresponding microwells. An outlet of the droplet generator is to be fluidly coupled to the inlet of the droplet loading device.

### RELATED APPLICATION

This application claims the benefit of and priority to U.S. Provisional Patent Application No. 63/458,869, filed Apr. 12, 2023, the content of which is incorporated by reference herein in its entireties and for all purposes.

### STATEMENT OF GOVERNMENT SUPPORT

This invention was made with government support under CA203018 awarded by the National Institutes of Health. The government has certain rights in the invention.

### BACKGROUND

Single-cell analyses are revolutionizing biomedicine and biology, with genomics (DNA) and transcriptomics (RNA) tools leading the way. At the protein level, single-cell analyses are limited to mass spectrometry and immunoassays. Neither assay provides comprehensive coverage of proteome for single cells, missing key proteoforms (including isoforms, splice variants, and combinations of post-translational modifications).

### SUMMARY

Shortcomings of the prior art can be overcome and advantages and benefits as described later in this disclosure can be achieved through the provision of droplet-electrophoresis devices and related methods. Various implementations of the apparatus and methods are described below, and the apparatus and methods, including and excluding the additional implementations enumerated below, in any combination (provided these combinations are not inconsistent), may overcome these shortcomings and achieve the advantages and benefits described herein.

The disclosed examples allow simultaneous workflow for single cell isolation, encapsulation, lysis, and subsequent immunoblotting to determine the presence of antigens present in, for example, a tumor cell as an example. The disclosed examples allow isoforms of those antigens to be identified as an example. Among different samples, different expression levels as measured by intensity exist and different patterns of expression exist.

In accordance with a first implementation, an apparatus includes a droplet generator, a housing, a microwell assembly, and a droplet loading device. The droplet generator includes an oil fluidic line, a sample fluidic line, a buffer fluidic line, a droplet generation area, and an outlet. The oil fluidic line includes an oil inlet and an oil outlet, the sample fluidic line includes a sample inlet and a sample outlet, and the buffer fluidic line includes a buffer inlet and a buffer outlet. The droplet generation area is where the oil outlet, the sample outlet, and the buffer outlet are coupled and the outlet is fluidly coupled to the droplet generation area. The housing includes a perimeter wall including an interior side wall and a lip that define a receptacle. The microwell assembly is positioned within the receptacle and rests on the lip. The microwell assembly includes a plurality of microwells. The droplet loading device is positioned within the receptacle on top of the microwell assembly. The droplet loading device includes an inlet manifold assembly including an inlet, an outlet manifold assembly including an outlet, and a plurality of channels fluidly coupled to the inlet manifold assembly and the outlet manifold assembly. Each of the channels have an opening positioned over top of corresponding microwells. The outlet of the droplet generator is to be fluidly coupled to the inlet of the droplet loading device.

In accordance with a second implementation, a method includes loading oil, a sample, and buffer into corresponding inlets of a droplet generator. The droplet generator includes an oil fluidic line including the oil inlet and an oil outlet, a sample fluidic line including the sample inlet and a sample outlet, a buffer fluidic line including the buffer inlet and a buffer outlet, a droplet generation area where the oil outlet, the sample outlet, and the buffer outlet are coupled, and an outlet fluidly coupled to the droplet generation area. The method includes generating droplets using the droplet generator that exit the outlet of the droplet generator and flowing the droplets to an inlet of an inlet manifold assembly of a droplet loading device positioned within a receptacle of a housing on top of a microwell assembly including a plurality of microwells. The droplet loading device includes an inlet manifold assembly including an inlet, an outlet manifold assembly including an outlet, and a plurality of channels fluidly coupled to the inlet manifold assembly and the outlet manifold assembly. Each of the channels have an opening positioned over top of corresponding microwells. The method includes directing the droplets to the channels using the inlet manifold assembly, flowing the droplets through the channels, and receiving the droplets in the corresponding microwells using gravity.

In further accordance with the foregoing first and/or second implementations, an apparatus and/or method may further include or comprise any one or more of the following:

In an implementation, oil, a sample, and buffer are to be loaded into the corresponding inlets of the droplet loading device. The droplet generator is to generate droplets that exit the outlet of the droplet generator.

In another implementation, the droplet loading device is to process the sample without substantial protein loss.

In another implementation, the oil includes mineral oil supplemented with Span 80 surfactant.

In another implementation, a concentration of the Span 80 surfactant is between about 0.2% and about 5%.

In another implementation, the buffer includes a Lysis buffer.

In another implementation, the Lysis buffer includes sodium dodecyl sulphate (SDS) in water.

In another implementation, a concentration of the sodium dodecyl sulphate is between about 0.1% and about 2%.

In another implementation, the droplet generator is to generate droplets that are substantially stable to enable cell lysis.

In another implementation, the droplets being substantially stable includes the droplets remaining intact without substantial expansion and without substantial shrinkage when exposed to temperatures between about 90° C. and about 100° C. and for incubation periods of between about 1 hour and about 2 hours.

In another implementation, the droplets have a diameter of between about 25 microns and about 95 microns.

In another implementation, a diameter of the droplets is about 5 microns less than a diameter of the corresponding microwells.

In another implementation, droplets that exit the outlet of the droplet generator are loaded into the inlet of the inlet manifold assembly to allow the droplets to flow through the channels and be received within corresponding microwells.

In another implementation, each of the channels has a height of between about 30 microns and about 100 microns.

In another implementation, the microwells each have a diameter of between about 30 microns and about 100 microns.

In another implementation, the perimeter wall includes an end. The housing includes a lid having a central opening that corresponds to the receptacle. The lid is to mate with the end of the perimeter wall.

In another implementation, the perimeter wall and the lid define corresponding fastener holes. The apparatus includes fasteners that extend through the fastener holes of the perimeter wall and the lid is to secure the lid to the perimeter wall.

In another implementation, the apparatus includes magnets. The perimeter wall includes an end that defines magnet receptacles and the magnets are to be positioned with the corresponding magnet receptacles.

In another implementation, the apparatus includes alignment pins. The perimeter wall includes transverse holes that extend through the perimeter wall and open into the receptacle and the alignment pins are to be positioned within corresponding transverse holes.

In another implementation, the alignment pins are to adjust the relative position of the droplet loading device to align the openings of the channels of the droplet loading device with the microwells of the microwell assembly.

In another implementation, the apparatus includes a tube to fluidly couple the outlet of the droplet generator to the inlet of the droplet loading device.

In another implementation, the microwell assembly includes a first layer, a second layer, and a third layer.

In another implementation, the first layer includes Polydimethylsiloxane, the second layer includes gel, and the third layer includes glass.

In another implementation, the gel includes a polyacrylamide concentration of between about 4% and about 10%.

In another implementation, the second layer is positioned between the first layer and the third layer.

In another implementation, the second layer defines the microwells.

In another implementation, the method includes performing an electrophoresis procedure on the microwell assembly.

In another implementation, the microwell assembly includes a first layer, a second layer including gel, and a third layer.

In another implementation, the method includes applying an electric current to the gel to perform an electrophoresis procedure.

In another implementation, generating the droplets using the droplet generator that exit the outlet of the droplet generator includes generating droplets that are substantially stable to enable cell lysis.

In another implementation, generating the droplets using the droplet generator that exit the outlet of the droplet generator includes generating droplets that remain intact without substantial expansion and without substantial shrinkage when exposed to temperatures between about 90° C. and about 100° C. and for incubation periods of between about 1 hour and about 2 hours.

In another implementation, the method includes adjusting a relative position of the droplet loading device using alignment pins to align the openings of the channels of the droplet loading device with the microwells of the microwell assembly.

In another implementation, the sample includes a frozen biopsy sample from a patient.

In another implementation, the sample includes cancer cells.

In another implementation, the sample includes non-cancer cells.

In another implementation, the sample includes at least approximately 10,000 cells.

It should be appreciated that all combinations of the foregoing concepts and additional concepts discussed in greater detail below (provided such concepts are not mutually inconsistent) are contemplated as being part of the subject matter disclosed herein and/or may be combined to achieve the particular benefits of a particular aspect. In particular, all combinations of claimed subject matter appearing at the end of this disclosure are contemplated as being part of the subject matter disclosed herein.

### BRIEF DESCRIPTION OF THE DRAWINGS

FIG. 1 illustrates a schematic diagram of an implementation of a system in accordance with the teachings of this disclosure.

FIG. 2 is a schematic illustration of an implementation of a droplet generator that can be used to implement the droplet generator of FIG. 1.

FIG. 3 is a detailed view of a first segment and a second segment of the inertial focusing portion of the droplet generator of FIG. 2.

FIG. 4 is a plan view of an implementation of a droplet loading device that can be used to implement the droplet loading device of FIG. 1.

FIG. 5 is an isometric expanded view of a housing including the receptacle and a lid, a microwell assembly, and a droplet loading device that can be used to implement the housing, the microwell assembly, and the droplet loading device of FIG. 1.

FIG. 6 is an isometric view of the housing, the microwell assembly, and the droplet loading device of FIG. 5.

FIG. 7 is an isometric view of a system including a droplet generator, a housing, a microwell assembly, and a droplet loading device that can be used to implement the droplet generator, the housing, the microwell assembly, and the droplet loading device of FIG. 1.

FIG. 8 is a cross-sectional view of an implementation of a microwell assembly that can be used to implement the microwell assembly of FIG. 1.

FIGS. 9a-9e show DropBlot: a hybrid droplet and single-cell protein electrophoresis bioMEMS device to understand the proteome of even rugged cell specimens; where FIG. 9a shows a workflow for single-cell electrophoresis using the integrated system. FIG. 9b shows a photo of the device assembles with droplet generation stage and all-in-one electrophoresis chamber. FIG. 9c shows a photo of polyacrylamide gel stippled with microwells, in which droplets are loaded. FIG. 9d shows fluorescence imaging of BSA at the 20 s lapsed separation time. Electric field: 40V/cm. FIG. 9e shows Background subtracted fluorescence intensities (AFU) of one separation lane. The insert is a bright-field image of the droplet after electrophoresis.

FIGS. 10a-10i show optimization of droplet stability to ensure complete cell lysis and reduce protein loss. FIG. 10a shows droplet generations with 0.5-2% (w/v) SDS in the disperse medium (Vdisperse: Vcontinuous=10:15 μL/min). The concentration of Span80 in continuous phase is 2% (v/v). FIG. 10b shows the stability of droplets (diameter: 50 μm) containing 0.5% SDS under a series of incubations. Top liquid layer (transparent): mineral oil; Bottom liquid layer (milky): droplets. FIG. 10c shows droplet enumeration after incubations (step1: 100° C. for 1h; step 2:100° C. for 1h; step 3:80° C. for 1h). Droplets were incubated and imaged on a glass slide with a hydrophobic surface. FIG. 10d shows in-droplet cell (MCF7) lysis with different concentrations of SDS at 95° C. Droplet diameter: 50 μm; FIG. 10e shows in-droplet cell (MCF7) lysis under room temperature using 0.5% SDS (w/v). FIG. 10f shows droplet insulation test in W/O droplet. (left): fluorescence image of droplet loaded with Alexa-Fluor 555 labeled BSA. (right): Mean fluorescence intensity of ~300 droplets over time (0-180 min). Droplets were individually loaded with AF488-IgG, AF555-BSA, AF647-OVA, and GFP. FIG. 10g shows mean fluorescence intensity of background over time (0-180 min). FIG. 10h shows mean fluorescence intensity of single droplets, loaded with AF555-BSA, at 0 min and 180 min. FIG. 10i shows mean fluorescence intensity of two adjacent droplets loaded with GFP (left, green) and AF555-IgG (red, right). D1-GFP: GFP intensity in droplet #1; D2-IgG: IgG intensity in droplet #2; D2-GFP: GFP intensity in droplet #2; D1-IgG: IgG intensity in droplet #1. FIGS. 11a-11h show systematic simulation and validation of prototype DropBlot devices with purified proteins. FIG. 11a shows a droplet stability test under electric field (field strength: 40 V/cm). The droplets remain intact after 120 s′ electrophoresis. FIG. 11b shows a top view of the 2D DropBlot model. Position of microwell center: X=0 μm. The Y position of droplet center is the same to that of microwell. FIG. 11c shows optimization of the electric potential and electric field. The red line represents electric field strength in the middle of separation lane along the x direction. FIG. 11d shows simulation of the migration distance (left) and concentration profiles (right) of BSA with different droplet positions. The microwell diameter was 50 μm and droplet diameter was 45 μm. The X position of droplet center ranged from −2.5 μm to 2.5 μm. σ: peak width (x) of protein band. Electric field strength: 40 V/cm; Electrophoresis time: 60 s. FIG. 11e shows immunofluorescence images of BSA (AF555 labeled) electromigration when droplet had different initial X position. Electric field strength: 40 V/cm; Electrophoresis time: 60 s. Microwell diameter: 50 μm; Droplet diameter: 45 μm. FIG. 11f show the simulation of effect of electrophoresis time (t: 0-60 s) and electric field strength (E: 20-60 V/cm) on the BSA electromigration. The droplet position is X=2.5 μm (right edge of microwell). FIG. 11h show imunofluorescence images (left) of BSA (AF555 labeled) electromigration at different electrophoresis time (t=15s, 30s, 45s, 60s). The line graph (right) represents the migration distance, and the error bar represents the peak width.

FIGS. 12a-12g show urified proteins separation using DropBlot with optimized conditions. Microwell diameter: 50 μm; Droplet diameter: 45 μm; Droplet position: X=2.5 μm. FIG. 12(a) Simulation of concentration profiles of BSA (top, pink) and OVA (bottom, blue) when E=40 V/cm, t=30s. FIG. 12(b) Simulation of BSA and OVA separation resolution at different electric field strength (E: 20-60 V/cm) and electrophoresis time (t: 0-60 s). The circles indicate that OVA touches the right edge of the migration lane. FIG. 12(c) Immunofluorescence image (top) and intensity profile (bottom) of BSA (AF555, pink) and OVA (AF647, blue) separation when the E=40 V/cm, t=30s. FIG. 12(d) Immunofluorescence image (top) and intensity profile (bottom) of BSA (AF555, pink) and IgG (AF488, blue) separation when the E=40 V/cm, t=30s. (e) Quantified migration distance (left) and separation resolution (right) from a total of n=500 lanes passing quality control in two different electrophoresis gels. E=40 V/cm, t=30s. (d) Immunofluorescence image (top) and intensity profile (bottom) of BSA (AF555, pink) and IgG (AF488, blue) separation when the E=40 V/cm, t=45s. (e) Quantified migration distance (left) and separation resolution (right) from a total of n=500 lanes passing quality control in two different electrophoresis gels. E=40 V/cm, t=45s.

FIGS. 13a-13h show the dropBlot device validation using fresh cancer cells. FIG. 13(a) shows representative EpCAM separations from MCF7 breast cancer cell lines lysed with different buffer formulations (0.5% SDS lysis buffer and 0.5% SDS lysis buffer supplemented with 6M urea), both after 30 s electrophoresis at an electric field strength of 40V/cm. FIG. 13b shows representative vimentin separations from MDA-MB-231 breast cancer cell lines lysed with different buffer formulations (0.5% SDS lysis buffer and 0.5% SDS lysis buffer including 6M urea), both after 30s electrophoresis at an electric field strength of 40V/cm. Immunofluorescence images of FIG. 13 shows MCF7 cells and FIG. 13d shows MDA-MB-231 cells after 30s electrophoresis at an electric field strength of 60V/cm. The lysis buffer was 0.5% SDS supplemented with 6M urea. Four channels were used in the immunoprobing, including the epithelial marker EpCAM (green), mesenchymal marker vimentin (red), human epidermal growth factor receptor 2 (HER2, cyan), and glycolytic enzyme GAPDH (blue). FIG. 13e shows Intensity profiles of proteins in a single migration lane of MCF7 (left) and MDA-MB-231 (right). FIG. 13f shows the migration distance of proteins in MCF7 (left) and MDA-MB-231 (right), n=1000. FIG. 13g shows the integrated peak area of each protein in MCF7 (left) and MDA-MB-231 (right), n=1000. FIG. 13h shows the peak width with respect to migration distance of proteins in MCF7 (left) and MDA-MB-231 (right), n=1000.

FIGS. 14a-14i show DropBlot device validation using fixed cancer cells. The cells were fixed with paraformaldehyde (PFA, FIGS. 14a-e) or methanol (FIGS. 14f-h). Cell loaden droplets were incubated at 98° C. for 60 min, and the electric field strength was 60 V/cm. Intensity plots (t=30s and 60s) and immunofluorescence images (t=60s) of proteins in PFA-fixed MCF7 cells shown in FIG. 14(a) and MDA-MB-231 cells shown in FIG. 14(b). The migration distance shown in FIG. 14(c) and peak intensity shown in FIG. 14(d) of EpCAM in MCF7 cells under different fixation and incubation conditions. Cells were fixed with PFA for 15-30 min, and the cell (30 min fixation) loaden droplets were incubated for 2 hours. FIG. 14e shows the integrated peak area of proteins in MDA-MB-231 cells that were fixed with PFA for different time (15 min and 30 min). FIG. 14f shows the intensity profiles of EpCAM in methanol-fixed MCF7 cells with different electrophoresis time (t=30s, 60s, 90s, and 120s). Immunofluorescence image (top panel) and intensity profile (bottom panel) of EpCAM in methanol-fixed MCF7 cells shown in FIG. 14g and vimentin in methanol-fixed MDA-MB-231cells shown in FIG. 14(h). the electrophoresis time was 120 s. FIG. 14i shows the integrated peak area of EpCAM in MCF7 cells (top) and vimentin in MDA-MB-231cells (bottom). The cell was fixed with PFA (fixation time, 15 min; incubation time, 1 h; electrophoresis time, 60 s) or Methanol (incubation time, 1 h; electrophoresis time, 120 s).

FIGS. 15a-15f show simulations of microwell diameters and thicknesses of the oil layer.

FIG. 16 shows a simulation of electromigration of BSA with different thicknesses of oil layer.

FIG. 17 shows electromigration of BSA with different relative positions of droplets (simulation). From the top to the bottom panel, the relative position of the droplet is right, middle and right to the microwell, respectively. The electric field strength is 40 V/cm and the electrophoresis time is 0-20 s.

FIGS. 18a-18c shows the limit of detection (LOD) of DropBlot on BSA analysis, where FIG. 18a shows the migration distance of BSA after 30 s′ electrophoresis with different protein mass per droplet (1620 fg, 330 fg, 66 fg, 13.2 fg, and 2.6 fg).

FIG. 19 shows an example implementation of a DropBlot holder and loading devices.

FIGS. 20a-20f discloses an application of DropBlot device on human tissue specimens from breast cancer patients. (a) Schematic illustration of clinical sample preparation for DropBlot. (b) Immunofluorescence images of EpCAM (Green, Alexa Fluor 488-labeled secondary antibody), Vimentin (Red, Alexa Fluor 594-labeled secondary antibody), and Her2 (Blue, Alexa Fluor 647-labeled secondary antibody), proteins retrieved from patient with triple positive breast cancer. (c) Mean intensity of EpCAM, Vimentin isoforms (Vimentin′, Vimentin″), and Her2 of cells (n=~1000) collected from breast cancer patient samples. Sample #1-2 were fresh cell suspensions. Sample #3-5 were fresh tissue. All the samples were fixed with 4% PFA. (d) Intensity of EpCAM, Vimentin isoforms (Vimentin′, Vimentin″), and Her2 of single cells (sample #3) in different microwells. (e) Immunofluorescence images of single migration lanes in sample #3. Green: EpCAM, Red: Vimentin. (f) Venn diagram depicts the percentage of proteins (EpCAM, Vimentin isoforms, Her2) in single cells of patient #3. Left: n=647, Right: n=516.

FIGS. 21a-21h discloses a DropBlot assay development utilizing single cells from two unfixed breast cancer cell lines, MCF7 and MDA-MB-231. (a) Fluorescence intensity profile for representative EpCAM PAGE separations from two unfixed MCF7 cells lysed with 0.5% SDS antigen-retrieval buffer without (top) and with (bottom) a 6 M urea supplement (DtPAGE=30 s; E=40 V/cm). Fluorescence intensity plot (right) shows Gaussian fitting assuming three overlapping EpCAM peaks in SDS+6 M urea antigen-retrieval buffer formulation. (b) Fluorescence intensity profile for representative VIM PAGE separations from two unfixed MDA-MB-231 cells lysed with 0.5% SDS antigen-retrieval buffer without (top) and with (bottom) a 6 M urea supplement (Dt_{PAGE}=30 s; E=40 V/cm). Fluorescence intensity plot (right) shows Gaussian fitting assuming three overlapping VIM peaks in SDS+6 M urea antigen-retrieval buffer formulation. Fluorescence micrographs for single-cell PAGE of (c) unfixed MCF7 and (d) unfixed MDA-MB-231 cells for the protein targets EpCAM (green), mesenchymal marker VIM (red), human epidermal growth factor receptor 2 (HER2, cyan), and glycolytic enzyme GAPDH (blue) (Dt_{PAGE}=30 s; E=60 V/cm). (e) Fluorescence intensity profiles for unfixed cells analyzed in (c) and (d). (f) Migration distance analysis of respective protein targets from single-cell PAGE analysis of MCF7 (left) and MDA-MB-231 cells (right) (n=1000 cells; Dt_{PAGE}=30 s; E=60 V/cm). (g) Fluorescence area under curve (AUC) for PAGE analyses from (f). (h) PAGE migration distance and peak width for PAGE analyses from (f).

FIG. 22 discloses intensity profiles of EpCAM (Green, MCF7) and Vimentin (Red, MDA-MB-231) when using an antigen-retrieval buffer containing 0.5% SDS only (top panel) and 0.5% SDS+6M urea (bottom panel), both after 30 s electrophoresis at an electric field strength of 40V/cm. Droplet diameter: 45 μm. Scale bar: 200 μm.

FIG. 23 discloses droplet enumeration after 1-hour incubations at 100° C. Droplets are loaded with 0.5% (w/v) SDS & 6 M Urea or 1% (w/v) SDS & 6 M Urea. Droplet diameter: 45 μm.

FIG. 24 discloses proteins analyzed in the DropBlot.

FIG. 25 discloses the identification of cell type based protein and proteoform markers detected by single-cell western blot (Δt_{PAGE}=30 s; E=60 V/cm). (a) Single-cell western blot micrographs report differential identification of peripheral blood mononuclear cells (PBMC) and H1299 (fresh) cells using CD45 and Vimentin markers, respectively. (b) Single-cell western blot micrographs report differential identification of MDA-MB-231 and fibroblast (fresh) cells using Vimentin and Fibroblast activation protein-alpha (FAP) markers, respectively.

FIG. 26 discloses the DropBlot analysis of single PFA- and methanol-fixed patient-derived dissociated cancer cells. (a) Schematic of clinical sample preparation workflow for DropBlot. PFA conditions: Δtfixation=15 min; Δtincubation=1.0 h at 98° C.; (b) Fluorescence micrographs of single-cell western blots of EpCAM (Green, AF488-labeled secondary antibody), VIM (Red, AF594-labeled secondary antibody), and HER2 (Blue, AF647-labeled secondary antibody) from PFA-fixed tumor cell. Tumor was classified as triple-positive breast cancer. (c) Mean fluorescence intensity of single-cell western blot analyses of PFA-fixed, patient-derived tumor cells for EpCAM, VIM proteoforms (VIM′, VIM″), and HER2. Samples #1-2 were fresh cell suspensions. Sample #3-5 were fresh dissociated tissues. The tumor cells were identified as EpCAM+ or HER2+. (d) Fluorescence micrographs of single-cell western blots of PFA-fixed cells from Sample #3 from (c), with HER2 (blue, AF647-labeled secondary antibody) and VIM (red, AF594-labeled secondary antibody). (e) Cell gating using DropBlot. HER2+positive cells were further classified based on the expression levels of VIM′ and VIM″. The protein target was considered as negative when the intensity was less than 4. (f) Cell gating using DropBlot. EpCAM+positive cells were further classified based on the expression levels of VIM′ and VIM″. The protein target was considered as negative when the intensity was less than 4. (g) Venn diagram reporting the single-cell target-expression profile for each of single PFA-fixed cells from Sample #1-5 in (c).

FIG. 27 discloses samples tested with DropBlot.

FIG. 28 discloses venn diagram reports showing the single-cell target-expression profile for PFA-fixed EpCAM-/HER2-cells (n=351) from Sample #3 in FIG. 26.

### DETAILED DESCRIPTION

Although the following text discloses a detailed description of implementations of methods, apparatuses and/or articles of manufacture, it should be understood that the legal scope of the property right is defined by the words of the claims set forth at the end of this patent. Accordingly, the following detailed description is to be construed as examples only and does not describe every possible implementation, as describing every possible implementation would be impractical, if not impossible. Numerous alternative implementations could be implemented, using either current technology or technology developed after the filing date of this patent. It is envisioned that such alternative implementations would still fall within the scope of the claims.

The present disclosure provides a hybrid droplet-electrophoresis device, termed “DropBlot”, to detect proteins from patient-derived tissue biospecimens relevant to clinical medicine and pathology. In some embodiments, the droplet-electrophoresis device takes advantage of water-in-oil (W/O) droplets to encapsulate single cells derived from chemically fixed tissues, thus providing a picoliter-volume reaction chamber in which said cells are lysed and subjected to harsh lysis conditions (100° C., 2 hours), as needed for fixed cells. Droplets remain intact under the electric field and protein isoforms are shown to electromigrate out of the droplet and into a microfluidic separation channel where protein sizing takes place via the action of electrophoresis in a photoactive polyacrylamide (PA) gel. In some embodiments, the droplet-electrophoresis device analyzes protein profiles without, with minimal, or with less sample loss and separate protein isoforms from fresh or chemical-fixed cell samples at single-cell resolution, enabling large cohort research to investigate countless human tissue specimens.

The disclosed implementations relate to droplet-electrophoresis devices that integrate droplet microfluidics with single-cell immunoblotting and can be used in numerous applications as described herein. Microanalytical tools underpin many of the single-cell genomic (DNA) and transcriptomic (RNA) advances of recent years. Similarly, microfluidic tools are playing a significant role in understanding single-cell level protein expression and function, and are critical for disease diagnostics. Proteins play a vital role in cell states and cellular heterogeneity. Despite their crucial importance in biological processes, proteins, especially in fixed biological samples, are difficult to analyze with high throughput and high sensitivity at the single-cell level by classic mass spectrometry (MS) and capillary electrophoresis, immunohistochemical staining, single-cell western blot method.

By way of example, in some embodiments the disclosed implementations relate to droplet-electrophoresis devices that integrate droplet microfluidics with single-cell immunoblotting and are used in:

A. Biospecimens and fixed cells/tissues which are a major part of the clinic-pathological workflow and are widely used in routine diagnostics. The fixed samples, especially the formalin-fixed and paraffin-embedded (FFPE), are directly linked to clinical records and other pathological data (e.g., mRNA sequencing). However, until now, the fixed samples are usually not accessible and/or difficult and challenging using traditional methods for proteomic analysis due to the crosslinking of proteins that prevent efficient protein extraction.

B. Cell-cell, cell-exosome, and cell-bacteria communication play vital roles in disease progression, such as cancer metastasis, cell migration, and sepsis. Investigation of the secretion or expression of proteins or chemokines during communication can help understand the underlying mechanism of human biology or diseases. However, conventional microfluidic technologies cannot simultaneously pair the target cells or particles, monitor the communication process, and resolve the secreted chemokines or proteins.

C. Timed dosing of cells with specific sequencing of chemicals or stimulus has great value in the study of therapeutic efficiency and cell potency (e.g., cytokine expression change in stimulated mesenchymal stromal cells). Understanding the proteins or chemokines profiling during the timed dosing can help evaluate the cell qualities and estimate the treatment prognosis.

D. Multimodal analysis at the single-cell level can illustrate the correlations between proteome and transcriptome and provide a comprehensive understanding of cellular mechanisms of human disease. However, the complicated setup, low sensitivity, inaccessibility to fixed samples limited the application of multi-modal analysis.

E. Concatenating with droplet systems (e.g., 10× Genomics) can integrate the proteome with transcriptome or genome analysis while dramatically reducing the cost of single-cell multiomics.

To analyze proteins in biospecimens, cell-cell communication, and time dosing with specific stimulus at single-cell level with high throughput and high recovery rate, the present disclosure provides a hybrid droplet-electrophoresis device (DropBlot) designed to support harsh-cell lysis conditions and, thus, applicability to all kinds of biospecimens preserved in clinical repositories. The DropBlot system can preserve all the cell lysate in the droplet, thus enabling us to perform multimodal analysis on proteome, transcriptome, and genome. The stability property of droplets in DropBlot makes them suitable for existing commercial droplet-based applications.

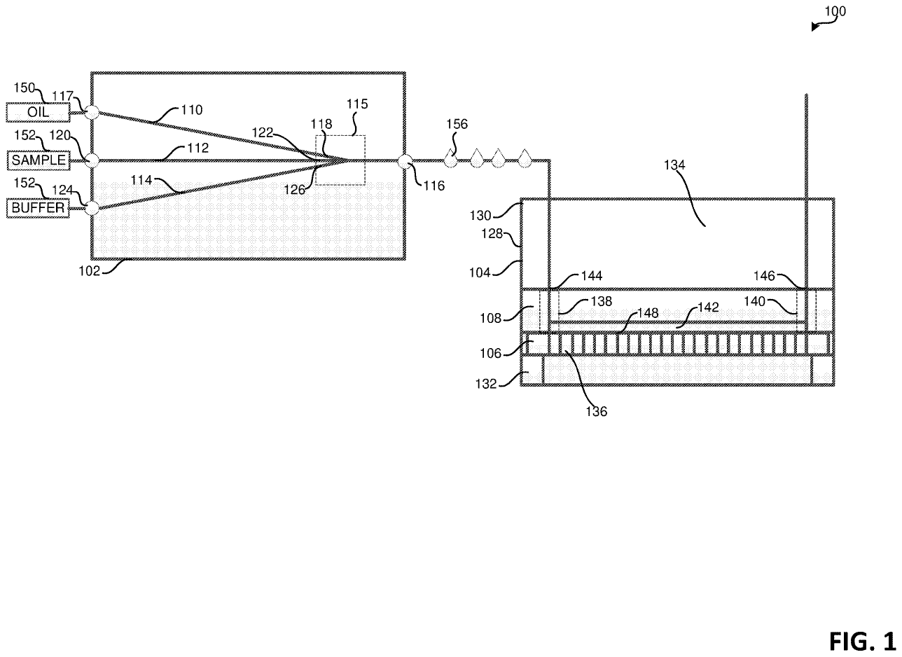

FIG. 1 is an implementation of an example system 100 that can be implemented as a hybrid droplet-electrophoresis device including a droplet generator 102, a housing 104, a microwell assembly 106, and a droplet loading device 108. The droplet generator 102 includes an oil fluidic line 110, a sample fluidic line 112, a buffer fluidic line 114, a droplet generation area 115, and an outlet 116. The oil fluidic line 110 may also be referred to as an immiscible phase fluidic line. The buffer fluidic line 114 may be referred to as a miscible phase fluidic line. The oil fluidic line 110 has an oil inlet 117 and an oil outlet 118, the sample fluidic line 112 has a sample inlet 120 and a sample outlet 122, and the buffer fluidic line 114 has a buffer inlet 124 and a buffer outlet 126. The droplet generation area 115 is where the oil outlet 118, the sample outlet 122, and the buffer outlet 126 are coupled and the outlet 116 of the droplet generator 102 is fluidly coupled to each of the droplet generation area 115. The outlet 116 is thus fluidly coupled to the oil outlet 118, the sample outlet 122, and the buffer outlet 126 in the implementation shown.

The housing 104 has a perimeter wall 128 having an interior side 130 and a lip 132 that define a receptacle 134. The microwell assembly 106 is positioned within the receptacle 134 and rests on the lip 132 and the droplet loading device 108 is positioned within the receptacle 134 on top of the microwell assembly 106. The microwell assembly 106 has a plurality of microwells 136 and the droplet loading device 108 has an inlet manifold assembly 138, an outlet manifold assembly 140, and a plurality of channels 142. The microwells 136 may be referred to as microscale wells, chambers, and/or compartments. The inlet manifold assembly 138 has an inlet 144 and the outlet manifold assembly 140 has an outlet 146, and the channels 142 are fluidly coupled to the inlet manifold assembly 138 and the outlet manifold assembly 140. Each of the channels 142 have an opening 148 positioned over top of corresponding microwells 136. The droplet loading device 108 may thus have an open microfluidic design and the channels 142 are shown open and are not enclosed. The outlet 116 of the droplet generator 102 is fluidly coupled to the inlet 144 of the droplet loading device 108 in the implementation shown. The microwell assembly 106 may include a gel such as Polyacrylamide gel.

Oil 150, a sample 152, and buffer 154 are loaded into the corresponding inlets 117, 120, 124 of the droplet loading device 108 in operation and the droplet generator 102 generates droplets 156 that exit the outlet 116 of the droplet generator 102. The sample 152 may include cells. The oil 150 may be implemented by other immiscible liquids, however. The buffer may be implemented by other miscible liquids such as aqueous solutions including buffers. The droplets 156 may be considered a reaction chamber that may be useful for (i) performing cell preparation (lysis at high temp with detergents), (ii) preparation of items that are not cells (organelles or other non-cell organisms such as algae or coral polyps) and/or (iii) other processes beyond sample prep (pairing of two interacting cells or a cell and perturbation; dosing a cell with a drug or other stimuli). The system 100 and/or the droplet loading device 108 may process the sample 152 without substantial protein loss in some implementations. The oil 150 may be a mineral oil supplemented with Span 80 surfactant as an example. A concentration of the Span 80 surfactant may be between about 0.2% and about 5%. The concentration of the Span 80 surfactant may different, however. The buffer 154 may be a Lysis buffer. The Lysis buffer may include sodium dodecyl sulphate (SDS) in water. A concentration of the sodium dodecyl sulphate may be between about 0.1% and about 2%. The Lysis buffer may include SDS and about 1x Tris-Glycine Buffer in water as an example. The concentration of the sodium dodecyl sulphate may be about 0.5%, for example.

The droplet generator 102 may generate droplets 156 that are substantially stable to enable cell lysis. The droplets 156 being substantially stable may include the droplets 156 remaining intact without substantial expansion and without substantial shrinkage when exposed to temperatures between about 50° C. and about 100° C. and/or between about 90° C. and about 100° C. and for incubation periods of between about 1 hour and about 2 hours and/or between about 0 hours and about 3 hours. As set forth herein, substantial expansion and substantial shrinkage of the droplet is around or is less than about 10% of the droplet diameter.

Droplets containing fresh cells may be incubated at around room temperature such as about 23° C. as an example. The sample 152 and the buffer 154 can be incorporated into the same droplet 156 using the droplet generator 102 and that droplet 156 may remain stable during high temperatures and longer incubation time periods. Droplets produced using the disclosed implementations may preserve all or substantially all of the cell lysate that may be extracted for downstream analysis.

The droplets 156 may have a diameter of between about 25 microns and about 95 microns in some implementations. The droplets 156 may have any diameter, however. A diameter of the droplets 156 may be about 5 microns less than a diameter of the corresponding microwells 136 as an example.

Droplets 156 that exit the outlet 116 of the droplet generator 102 are loaded into the inlet 144 of the inlet manifold assembly 138 in operation to allow the droplets 156 to flow through the channels 142 and be received within corresponding microwells 136. The microwells 136 may be open microwells (no lid) that are used to isolate and manipulate a single cell prior to lysis and protein electrophoresis. Each of the channels 142 may have a height of between about 30 microns and about 100 microns. The microwells 136 may each have a diameter of between about 30 microns and about 100 microns. One or more of the channels 142 may have the same or different heights than others of the channels 142 and/or may be a height outside of the about 30 microns and about 100 microns range.

The droplets 156 may be loaded into the microwells 136 based on a gravity. The sample 152 may be chemically lysed while encapsulated in the droplets 156, with each droplet 156 centered and/or positioned in a corresponding microwell 136. The droplet 156 may be enclosed in the oil 150 to inhibit protein lysate loss due to diffusion and substantially sustain stability of the droplet 156 under harsh lysis conditions. Harsh lysis conditions may include high temperatures and/or long incubation periods.

After the lysis processes are complete, protein analysis may be initiated by applying an electric field to the gel of the microwell assembly 106, causing protein electromigration out of the droplet 156 and into the gel molecular sieving matrix. Protein targets from the sample 152 separate in response to the electric field applied based on differences in electrophoretic mobility after protein electrophoresis initiates in a manner that is proportional to molecular mass or size of the protein targets. Resolved proteins may be covalently bonded to the gel of the microwell assembly 106 by UV-initiated capture and then labeled with fluorescent antibodies.

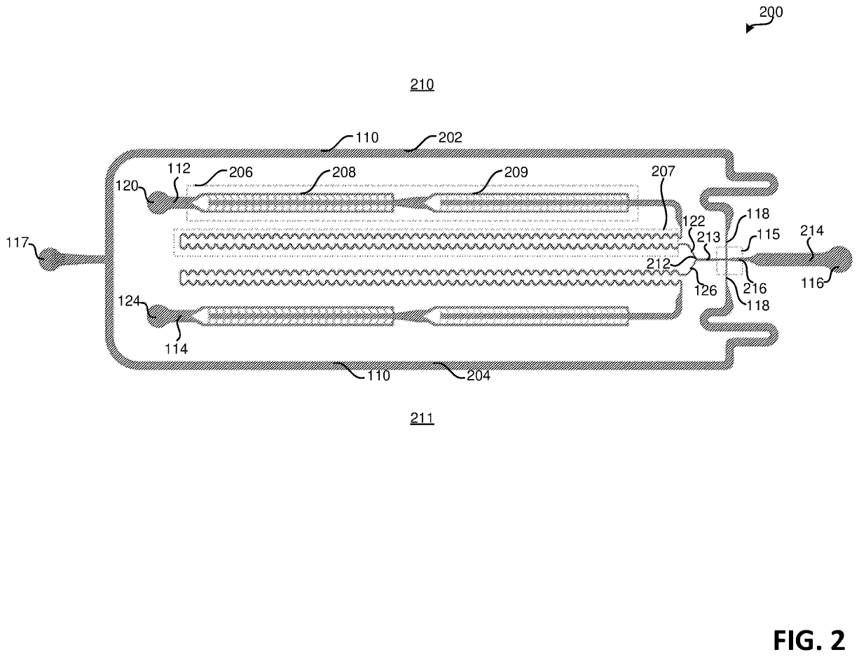

FIG. 2 is a schematic illustration of an implementation of a droplet generator 200 that can be used to implement the droplet generator 102 of FIG. 1. The droplet generator 200 includes the oil fluidic line 110 including a first oil fluidic line 202 and a second oil fluidic line 204, the sample fluidic line 112 including a debris removal portion 206 and an inertial focusing portion 207, and the buffer fluidic line 114. The debris removal portion 206 includes a first debris removal portion 208 and a second debris removal portion 209. The first debris removal portion 208 may include a top portion having between about 20 segments and about 30 segments, where the segments have a filter width of between about 50 microns and about 100 microns. The top portion of the first debris removal portion 208 may include 25 segments having a filter width of about 70 microns, for example. The top portion of the second debris removal portion 209 may include a top portion having between about 20 segments and about 35 segments, where the segments have a filter width of between about 50 microns and about 100 microns. The second debris removal portion 209 may include 28 segments having a filter width of about 30 microns, for example. The inertial focusing portion 207 may enable a flow rate of between about 5 microliters per minute to about 100 microliters per minute and/or include a top portion having between about 40 segments and about 60 segments, and have a channel height of between about 30 microns and about 90 microns. The inertial focusing portion 207 may include a top portion having 54 segments, have a channel height of about 60 microns, and enable a flow rate of about 10 microliters per minute, for example.

The flow rate of the oil 150 through the first oil fluidic line 202 and the second oil fluidic line 204 may be between about 0.02 microliters per minute and about 500 microliters per minute, the flow rate of the sample 152 through the sample fluidic line 112 may be between about 0.01 microliters per minute and about 100 microliters per minute, and the flow rate of the buffer 154 through the buffer fluidic line 114 may be between about 0.01 microliters per minute to about 100 microliters per minute.

The first oil fluidic line 202 and the second oil fluidic line 204 are both fluidly coupled to the oil inlet 117 and surround the sample fluidic line 112 and the buffer fluidic line 114. The first oil fluidic line 202 goes about the sample fluidic line 112 and the buffer fluidic line 114 on a first side 210 of the droplet generator 200 and the second oil fluidic line 204 goes about the sample fluidic line 112 and the buffer fluidic line 114 on a second side 211 of the droplet generator 200.

The outlets 118, 122, 126 of the first oil fluidic line 202, the second oil fluidic line 204, the sample fluidic line 112, and the buffer fluidic line 114 are coupled at the droplet generation area 115. The sample fluidic line 112 and the buffer fluidic line 114 are shown being coupled at an area 212 upstream of the droplet generation area 115 to allow the outlets 122, 126 of the sample fluidic line 112 and the buffer fluidic line 114 to be coupled to the droplet generation area 115. The area 212 is coupled to the droplet generation area 115 by a fluidic line 213 and the outlet 116 is coupled to the droplet generation area 115 by a fluidic line 214 in the implementation shown. A portion 216 of the fluidic line 214 may have a width of between about 40 microns and about 60 microns. The portion 216 may have a width of about 50 microns, for example.

FIG. 3 is a detailed view of a first segment 218 and a second segment 220 of the inertial focusing portion 207 of the droplet generator 200 of FIG. FIG. 2. The segments 218, 220 have a height 222 and a width 224. The height 222 may be between about 60 microns and about 120 microns and the width 224 may be between about 180 microns and about 360 microns. The height 222 may be about 100 microns and the width 224 may be about 300 microns as an example. An area 226 between the first segment 218 and the second segment 220 has a height 228 and a width 230. The height 228 may be between about 30 microns and about 60 microns and the width 230 may be between about 60 microns and about 120 microns. The height 228 may be about 50 microns and the width 230 may be about 100 microns, for example.

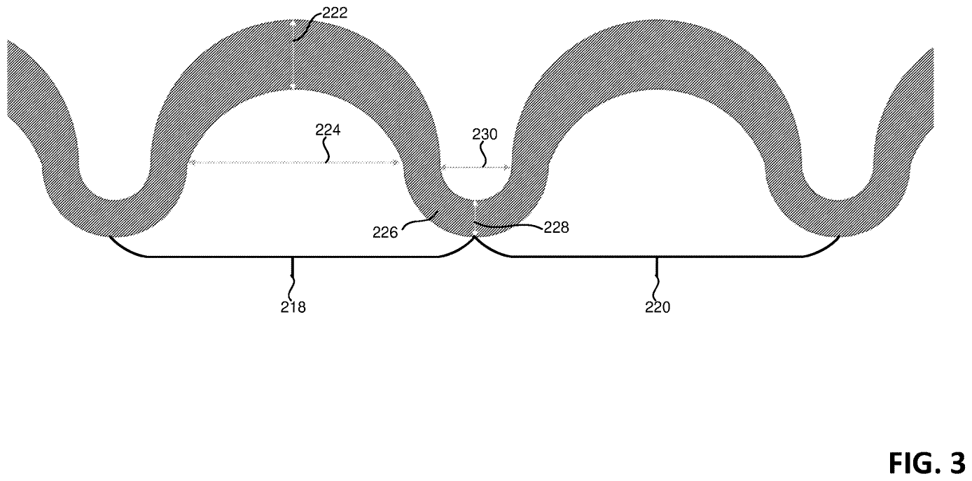

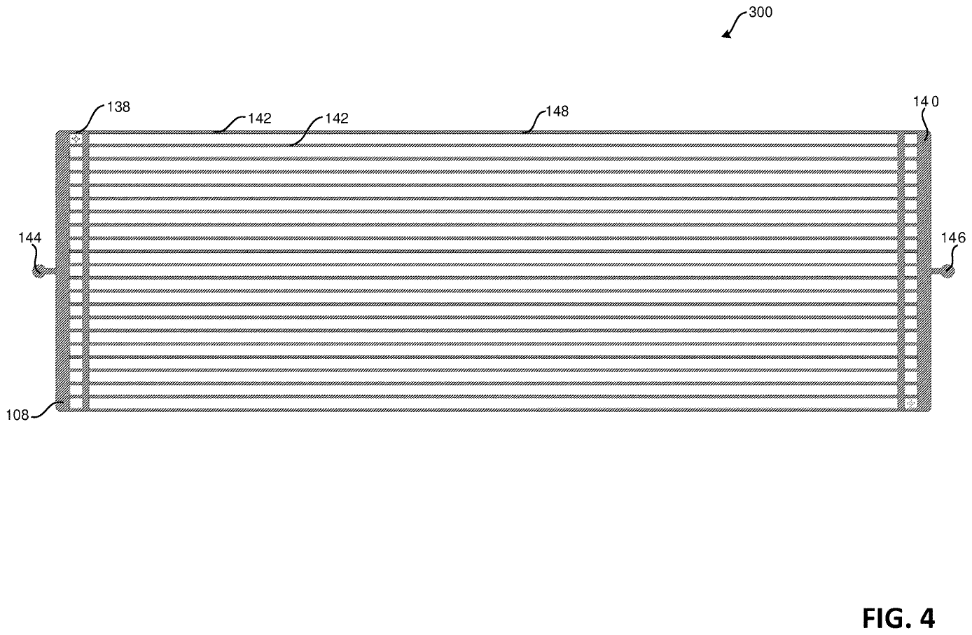

FIG. 4 is a plan view of an implementation of a droplet loading device 300 that can be used to implement the droplet loading device 108 of FIG. 1. The droplet loading device 300 includes the inlet manifold assembly 138 having the inlet 144, the outlet manifold assembly 140 having the outlet 146, and the channels 142. The channels 142 are coupled between the inlet manifold assembly 138 and the outlet manifold assembly 140.

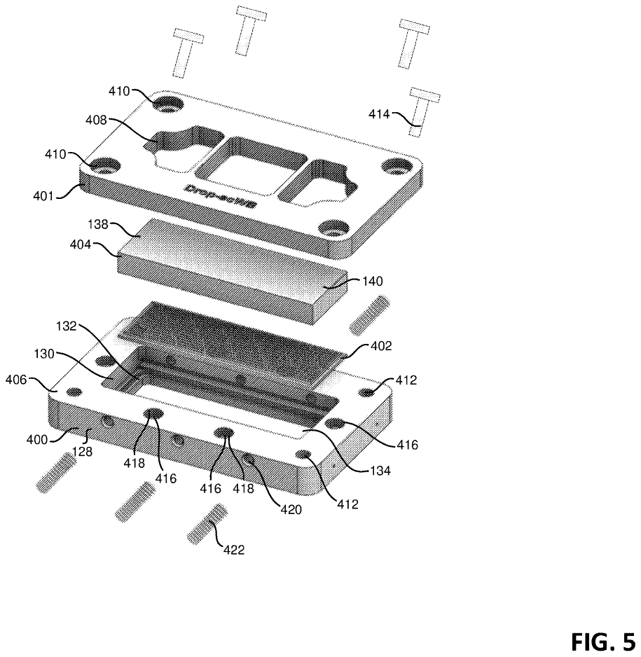

FIG. 5 is an isometric expanded view of a housing 400 having the receptacle 134 and a lid 401, a microwell assembly 402, and a droplet loading device 404 that can be used to implement the housing 104, the microwell assembly 106, and the droplet loading device 108 of FIG. 1. The housing 400 includes the perimeter wall 128 having an end 406 and the lid 401 mates with the end 406 of the perimeter wall 128. The lid 401 has a central opening 408 that corresponds to the receptacle 134 in the implementation shown.

The perimeter wall 128 and the lid 401 define corresponding fastener holes 410, 412 and fasteners 414 can extend through the fastener holes 410, 412 of the perimeter wall 128 and the lid 401 to secure the lid 401 to the perimeter wall 128. The end 406 of the perimeter wall 128 defines magnet receptacle 416 and magnets 418 are positioned with the corresponding magnet receptacle 416 in the implementation shown. The lid 401 may carry and/or include a ferrous material and/or a ferromagnetic material that is attracted to the magnets 418. The magnet receptacles 416 and/or the magnets 418 may be omitted, however.

The perimeter wall 128 is shown including transverse holes 420 that extend through the perimeter wall 128 and open into the receptacle 134 and alignment pins 422 can be positioned within corresponding transverse holes 420. The alignment pins 422 may adjust the relative position of the droplet loading device 108 to align the openings 148 of the channels 142 of the droplet loading device 108 with the microwells 136 of the microwell assembly 106. The droplets 156 can thus exit the bottom openings 148 of the channels 142 of the droplet loading device 108 and be loaded into the microwells 136 below.

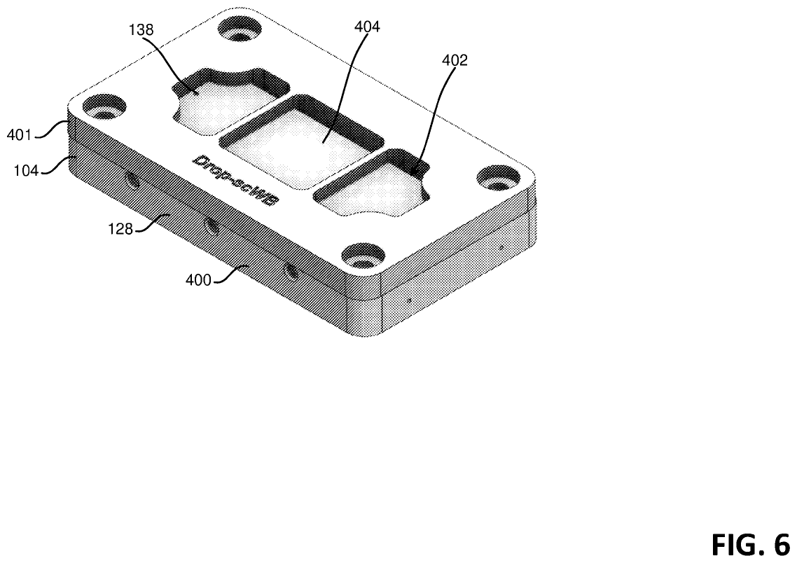

FIG. 6 is an isometric view of the housing 400, the microwell assembly 402, and the droplet loading device 404 of FIG. 5. The microwell assembly 402 and the droplet loading device 404 of FIG. 5 are shown positioned within the housing 400.

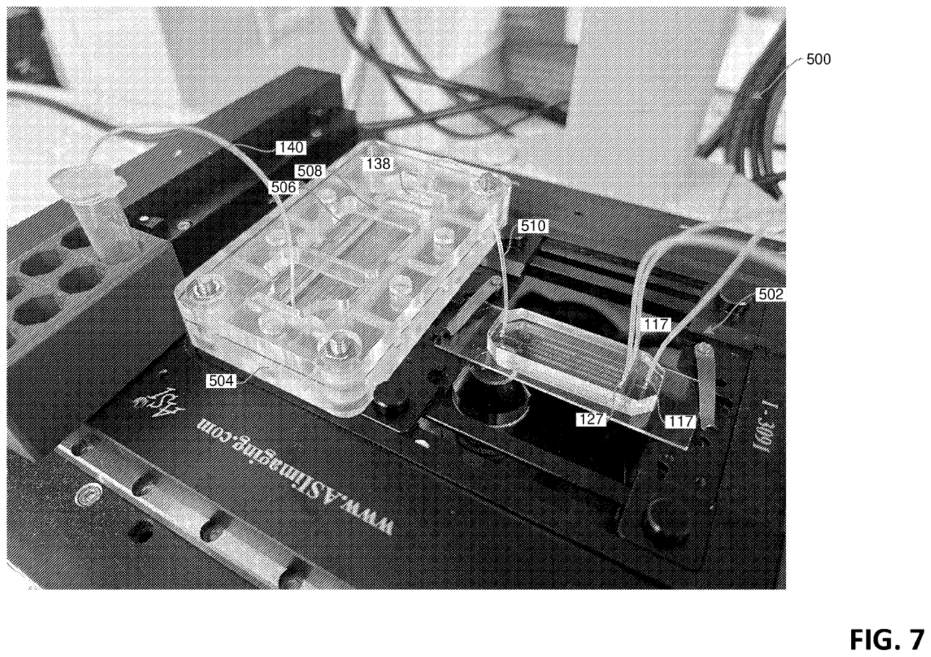

FIG. 7 is an isometric view of a system 500 including a droplet generator 502, a housing 504, a microwell assembly 506, and a droplet loading device 508 that can be used to implement the droplet generator 102, the housing 104, the microwell assembly 106, and the droplet loading device 108 of FIG. 1. A tube 510 is shown fluidly coupling the outlet 116 of the droplet generator 502 to the inlet 144 of the droplet loading device 508.

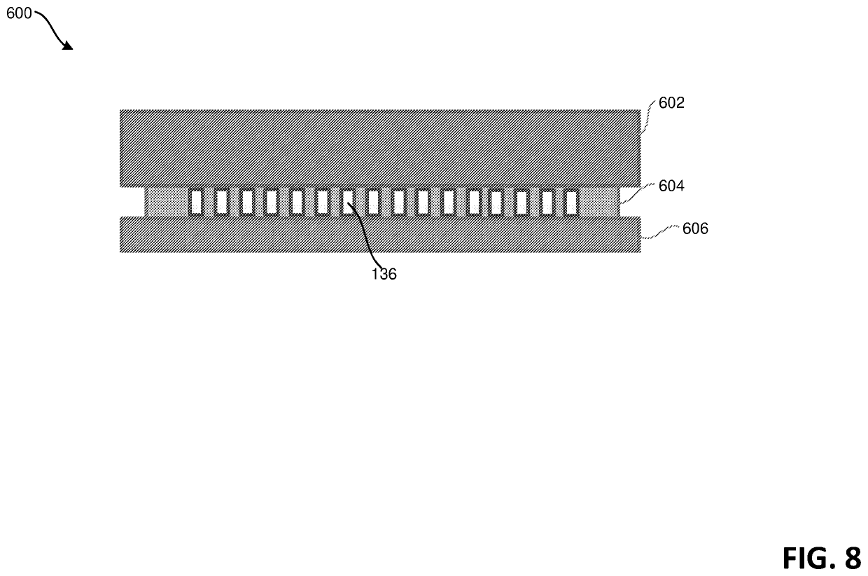

FIG. 8 is a cross-sectional view of an implementation of a microwell assembly 600 that can be used to implement the microwell assembly 106 of FIG. 1. The microwell assembly 600 includes a first layer 602, a second layer 604, and a third layer 606. The first layer 602 may include Polydimethylsiloxane, the second layer 604 may include gel, and the third layer 606 may include glass. The gel may have a polyacrylamide concentration of between about 4% and about 10%. The second layer 604 is positioned between the first layer 602 and the third layer 606 and the second layer 604 defines the microwells 136.

The following Example provides one embodiment of a hybrid droplet-electrophoresis device, referred to throughout the Example as “DropBlot”. Additional embodiments are provided herein and in the Figures.

### Example 1

### DropBlot Design Integrates Droplet Microfluidics with Single-Cell Electrophoresis for Targeted Proteomics

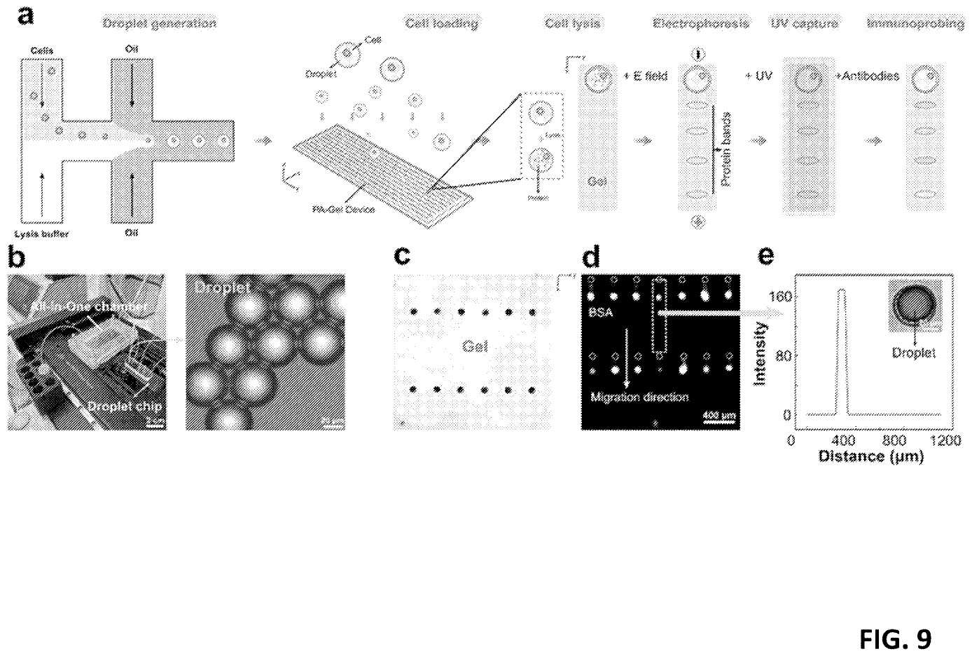

Overview of DropBlot. DropBlot incorporates a droplet generation device with a single-cell immunoblotting chamber, on which droplet trapping, cell lysis, on-chip protein separation, and antigen probing, are performed. In this device (FIG. 9a) cell suspensions and lysis buffer are encapsulated into water-in-oil (W/O) droplets. One of the major challenges in this system is to ensure one droplet encapsulates a single cell. To achieve this goal, a systematic optimization is performed by changing the channel geometry, flow rates, and initial cell concentration. The droplet generation device can produce monodisperse droplets that have a size range of 40-60 μm in diameter with a flow rate of 1-20 L/min. Initial cell concentration is adjusted to about 4e6 cells/mL to achieve a single-cell loading efficiency of 79%. The cell-laden droplets are then loaded onto a polyacrylamide gel (PA-gel) slide stippled with microwells via gravitational. About 5000 microwells are patterned on the PA-gel device with a pitch distance of 1000 μm in the x direction and 300 μm in the y direction. The cell in the droplet starts to be lysed as soon as a new droplet is generated and the lysis time will be various for different cell types and fixation conditions. The droplet, enclosed by mineral oil, prevents protein lysate loss due to diffusion and sustains stability under harsh lysis conditions (temperature: >95° C.; incubation time: >60 min). After the harsh lysis step completes, protein analysis is initiated by applying an electric field to the PA gel and droplet system, causing protein electromigration out of the W/O droplet and into the PA-gel molecular sieving matrix. Once protein electrophoresis initiates, protein targets separate based on differences in electrophoretic mobility (proportional to molecular mass or ‘size’). Resolved proteins are then covalently bonded to the gel by UV-initiated photocapture and then labeled with fluorescent antibodies. Based on the working principle, an All-in-One reaction chamber (FIG. 9b) was designed, on which the droplets loading, protein electrophoresis, and immunoblotting are carried out. Droplets in this system remain intact after protein electrophoresis. DropBlot can resolve proteins while maintaining the stability of droplets.

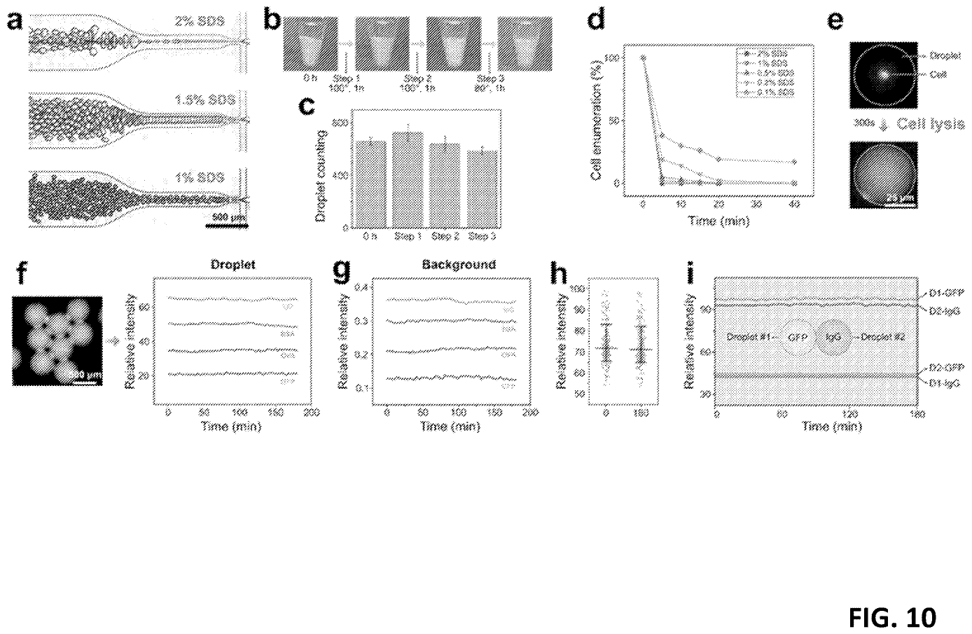

Droplet Stability Study and In-Droplet Cell Lysis. Because intact droplets can preserve all the cell lysate without protein loss, droplet stability plays a vital role in protein analysis using the DropBlot system. The droplet stability is determined by two major factors, including properties of surfactant used in the continuous phase (mineral oil in this study) 1,2, and components of the dispersed phase (lysis buffer and cell suspension) 3,4. Surfactants can prevent droplets from coalescing by reducing the interfacial tension between the continuous phase and surfactant, and help stabilized the droplet for long-term storage. Mineral oil supplemented with 0.5-5% (v/v) of Span80 is a well-established recipe to generate stable water-in-oil emulsion^{5,6}. However, the stability of droplets is also affected by the concentration of sodium dodecyl sulfate (SDS) used in the lysis buffer (dispersed phase). As is shown in FIG. 10a, increasing SDS concentration in the lysis buffer negatively correlates to droplet stability when the flow rate of disperse phase, V_{disperse}, is 10 μL/min and the flow rate of the continuous phase, V continuous is 15 μL/min, due to the destabilization effect of hydrophilic surfactants in the disperse phase⁴. A high concentration of SDS (e.g., 2%, w/v) requires a low flow rate to ensure SDS have enough time to redistribute near the water-oil interface which will sacrifice the throughput. Typically, antigen retrieval from fixed cell samples requires a long time (>60 min) and high temperature (>95° C.) incubation, which may induce the coalescence and breakage of the droplets. The stability of the droplets (size: 50 μm, Span80: 2% (v/v), SDS: 0.5% (w/v)) was tested by a series of incubation under harsh conditions according to two basic criteria: layer separation and droplet number. Stable droplets collection will maintain two layers (top: mineral oil, bottom: droplets). Once droplet breakage happens, there will be three layers co-exist in the collection tube (top: mineral oil, middle: droplets, bottom: lysis buffer). There is no obvious layer separation for 3 hours incubation at 80-100° C. (FIG. 10b) while the number of droplets has no remarkable change (FIG. 10c), indicating the high stability of droplets.

The in-droplet cell lysis efficiency was next tested using human breast cancer cell line GFP-MCF7 with lysis buffer containing 0.1%-2% (w/v) SDS (FIG. 10d). Lysis buffer with 2% (w/v) SDS can lyse cells within 5 minutes while the buffer with 0.1% SDS cannot completely lyse cells and about 25% of cells remain intact after 40 minutes of incubation. Having considered the negative effect of high concentration of SDS on the droplet stability and droplet generation throughput, 0.5% (w/v) is the ideal concentration of SDS that can completely lyse the cell within 15 min at room temperature (FIG. 10e) while maintaining high stability and throughput (>5 μL/min).

Droplet Sealing Study with Protein Ladder. The W/O droplets act as a closed reaction chamber for the cell lysis. To measure the protein-permeability of these droplets, a series of known protein standards was loaded and mixed with lysis buffer (SDS: 0.5%, w/v) into droplets and measured the mean fluorescence intensities of individual proteins (Immunoglobin (IgG, 150 kDa) labeled with AF488, Bovine Serum Albumin (BSA, 66kDA) labeled with AF555, Ovalbumin (OVA, 45 kDa) labeled with AF647 and Green Fluorescent Protein (GFP, 26 kDa)). As is shown in FIG. 10f, there was no obvious fluorescence intensity change during the 180-min assay (room temperature). Meanwhile, the intensity of the background did not increase over time (FIG. 10g). The mean intensity of individual droplets were monitored and it was found that the intensity distribution remains the same before (0 min) and after (180 min) experiment (FIG. 10h). W/O droplets used in the DropBlot assay are stable and can preserve all the proteins, no matter what the molecular weight they are, during a 180-min experiment. To investigate the crosstalk between adjacent droplets, the droplets loaded with Green Fluorescent Protein (GFP) and Immunoglobulin (IgG, AF555) were mixed and analyzed the fluorescence intensities for 180 min. The protein intensities remained stable during the assay and there was not crosstalk between neighboring droplets (FIG. 10i). Taken together, these experimental data show that droplets in this assay have good insulation performance and it can preserve the protein molecules without loss or exchange.

Simulation and Systematic Optimization of DropBlot. Experimental results, in FIG. 11a, show that the droplets remain intact during the electrophoresis (electric field strength: 40 V/cm, electrophoresis time: 120s). Given the integrated microfluidic design with droplet and polyacrylamide gel on the same chip, the protein migration mechanism under the current setup and the physics-based factors that set the highest protein separation resolution were investigated. The protein electromigration in this integrated system is mainly determined by electric field strength, microwell size, oil layer thickness, and the relative position between droplet and microwell. To investigate the migration mechanism and optimize the separation, a 2D model (FIG. 11b) was designed and simulated the protein movement on the DropBlot device.

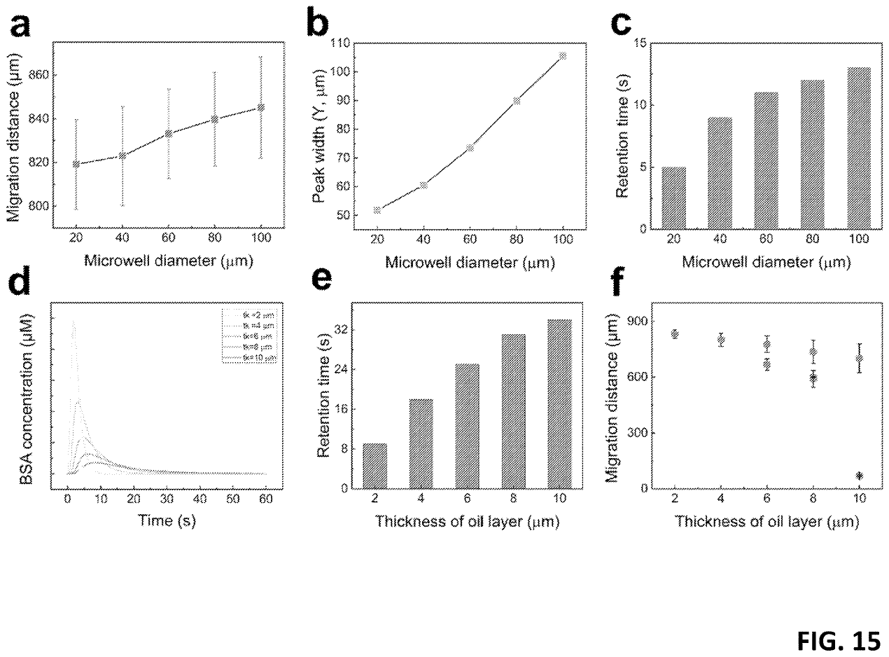

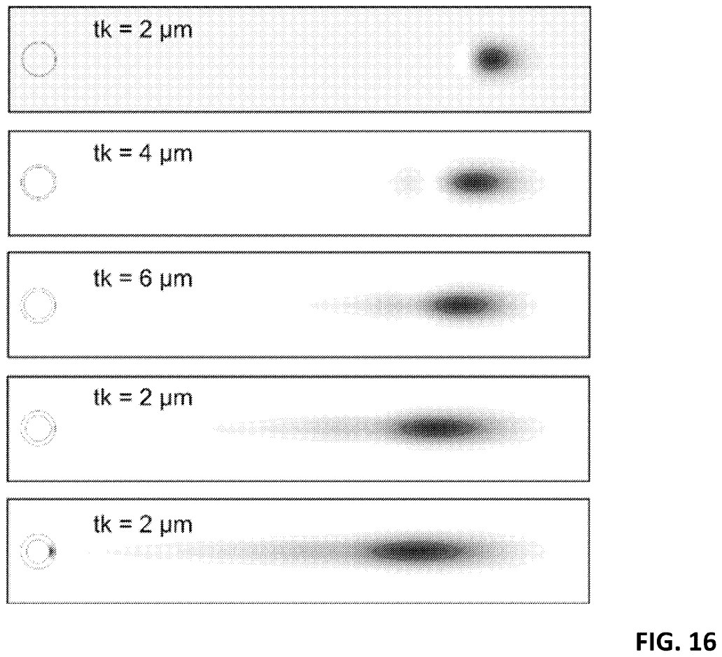

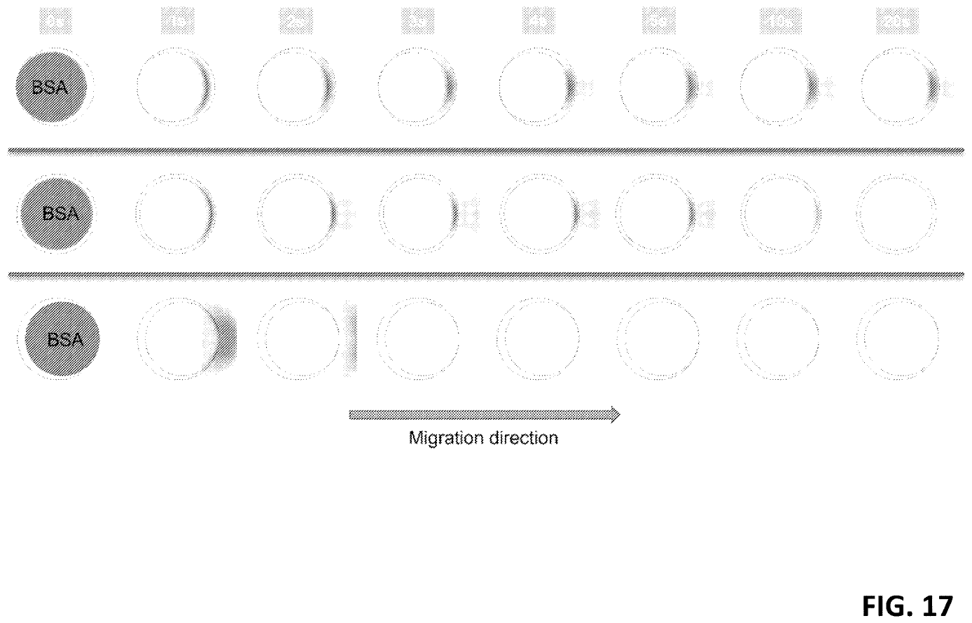

As is shown in FIG. 11c, the electric field strength increases near the outside edge of the microwell while decreasing in the oil layer (the region between the microwell and droplet), due to the low electrical conductivity (0.175 S/m) of mineral oil used in this assay. During the electromigration, proteins in the droplet would quickly accumulate near the right edge of the droplet and then slowly move through the oil region, which is the rate determining step. As such, the thickness of the oil layer would significantly affect the protein intensity profiles and migration distances. Here the oil layer is defined as the region between right edge of droplet and right edge of microwell when the protein migration direction is +X (from left to right, Y=0 μm). When the microwell size is constant, the thickness of oil layer is determined by droplet size and relative position of droplet in the microwell. The retention time, t_{RT}, in which protein molecules migrate through the oil region, is determined by the oil layer thickness and can be estimated based on equation (1).

$${t}_{RT} = \frac{{th}_{oil}}{{E}_{oil} \times {μ}_{EM \_ oil}} \qquad ( 1 )$$

Where thₒᵢₗ is the thickness of oil layer, Eₒᵢₗ is the electric field strength, and the μ_{EM_oil} is the electrophoretic mobility of protein molecules in the oil region. Increasing the thickness of oil layer, by reducing the droplet size or moving the droplet from X=−2.5 μm to X=2.5 μm., will induce longer retention time that the proteins stay in the microwell and broadened protein bandwidth (FIG. 11d, FIG. 15). The electrophoretic mobilities in the oil region were be determined to be (μ_{BSA}=125 μm²/(V·s)) based on experimental results (FIG. 11h). This simulation was verified with the experimental results using purified protein (BSA, FIG. 11e). Droplet positions can be adjusted using the gravity force by titling the all-in-one holder. As the droplet moves from left to the right side of the microwell, thickness of oil layer reduces and the peak width decreases. (FIG. 16). The peak width, σ, can be calculated based on the thickness of oil layer and diffusion coefficients of protein molecule in oil region and gel region:

$${σ}^{2} = {σ}_{0}^{2} + 2 {D}_{oil} {t}_{RT} + 2 {D}_{gel} {t}_{gel} \qquad ( 2 )$$

Where σ₀, is injected peak width that is determined by the retention time (t_{RT}), electric field strength in gel region (E_{gel}), and electrophoretic mobility (μ_{EM_gel}) of protein molecules in the gel region (eqn (3)). Dₒᵢₗ and D_{gel} are diffusion coefficients of protein in oil and gel region, respectively. t_{gel} is the diffusion time of protein in the gel region. The protein diffusion inside the droplet region is negligible, as proteins can accumulate near the right edge of the droplet within 1s (FIG. 17).

$${σ}_{0} = {t}_{RT} \times {E}_{gel} \times {μ}_{EM \_ gel} \qquad ( 3 )$$

Taken together eqn (1-3), the peak width of protein molecules in DropBlot can be obtained:

$${σ}^{2} = {( \frac{{th}_{oil}}{{E}_{oil} \times {μ}_{EM \_ oil}} \times {E}_{gel} \times {μ}_{EM \_ gel} )}^{2} + \frac{2 {D}_{oil} \times {th}_{oil}}{{E}_{oil} \times {μ}_{EM \_ oil}} + 2 {D}_{gel} \times ( t - \frac{{th}_{oil}}{{E}_{oil} \times {μ}_{EM \_ oil}} ) \qquad ( 4 )$$

By increasing droplet size and shifting droplet to the right edge of microwell, the thickness of oil layer can be reduced and thus minimizing the peak width.

In addition, the peak profiles of proteins can be affected by the microwell size and peak width in the Y direction will be increased. Considering the size (45 μm) of stable droplet produced droplet and different loading efficiency for different microwell size in experiments, a 50 μm microwell patterns was used and ‘droplet-right-edge’ position in the following simulations and experiments. Furthermore, the effect of electrophoresis time on the migration distance was accessed. The migration distance is proportional to the electrophoresis time and electric field strength (FIG. 10f). BSA may be used to validate the simulation result. The protein profiles (FIG. 10h) further confirmed that constant velocity of protein in the gel matrix and the approximate electrophoretic mobility of BSA is about 3590 μm²/(V·s),. The BSA migration distance is directly proportional to the electrophoresis time (EF: 40 V/cm, FIG. 10h).

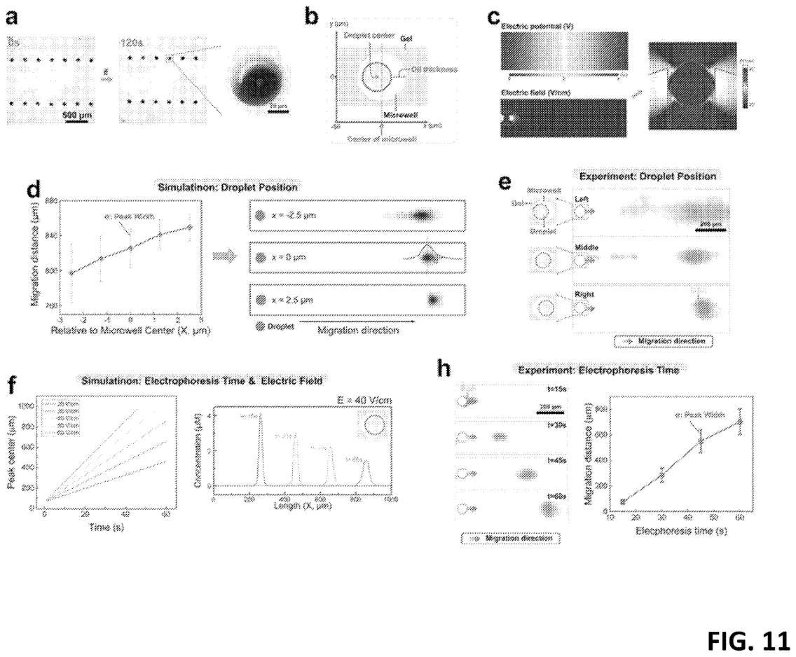

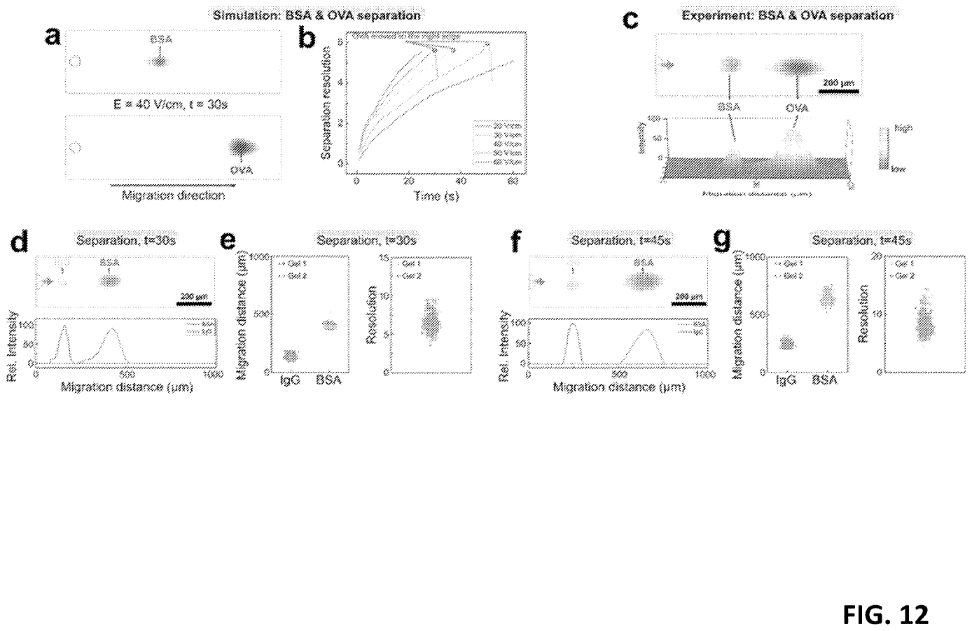

Application of DropBlot to Purified Proteins Separation. The optimized conditions (Diameter of microwell: 50 μm, Diameter of droplet: 45 μm, droplet position: right edge) were used to perform DropBlot assay and develop a model to provide guidance in the protein separation (BSA and OVA (ovalbumin)). The OVA (MW, 43 kDa) and BSA (MW, 66 kDa) can be completely separated after 30 s′ electrophoresis with an electric field strength of 40 V/cm (FIG. 12a). The protein migration during gel electrophoresis is determined by the molecular weight: proteins with lower molecular weight move faster than the one with high molecular weight.¹ The separation resolution is related to the electrophoresis time and electric field strength (FIG. 12b). The separation resolution can be calculated as follows:²

$$R = \frac{Δ x}{\frac{1}{2} \times ( 4 {σ}_{1} + 4 {σ}_{2} )} = \frac{{Δμ}_{EM \_ oil} \times {E}_{oil} \times {t}_{RT} + {Δμ}_{EM \_ gel} \times {E}_{gel} \times ( t - {t}_{RT} )}{\frac{1}{2} \times ( 4 {σ}_{1} + 4 {σ}_{2} )} \qquad ( 5 )$$

where Δx is the difference in migration distance between two types of proteins, σ₁, and σ₂ is peak width of the protein targets, Δμ_{EM_oil} and Δμ_{EM_gel} are difference in electrophoretic mobility between two types of proteins in the oil region and gel region, respectively. The diffusion coefficients of BSA and OVA were estimated based on Stoke-Einstein equation. 3 The value in the oil region is about 2.40 μm²/s and 3.23 μm²/s at room temperature, respectively. The value in the gel region is about 3.45 μm²/s and 4.65 μm²/s at room temperature, respectively. BSA and OVA proteins were diluted to 0.1 mg/ml and encapsulate in in 45 μm droplets containing 0.5% (w/v) SDS. The electrophoretic mobilities in the 8% T gel were be determined to be (UBSA=3590 μm²/(V·s), μ_{OVA}=5830 μm²/(V·s)) based on experimental results (FIG. 11h). The separation resolution is proportional to the electric field strength and electrophoresis time. In this work, E of 20-60 V/cm was mainly used, because the separation resolution can be reduced when using higher electric field strength due to the Joule heating.⁴ To validate this simulation, DropBlot was used to separate BSA and OVA proteins. The BSA and OVA can be completely separated after 30 s′ electrophoresis when the electric field strength is 40 V/cm (FIG. 4c). The separation resolution is about 3.8, which is smaller than the simulation result (R=4.9). T

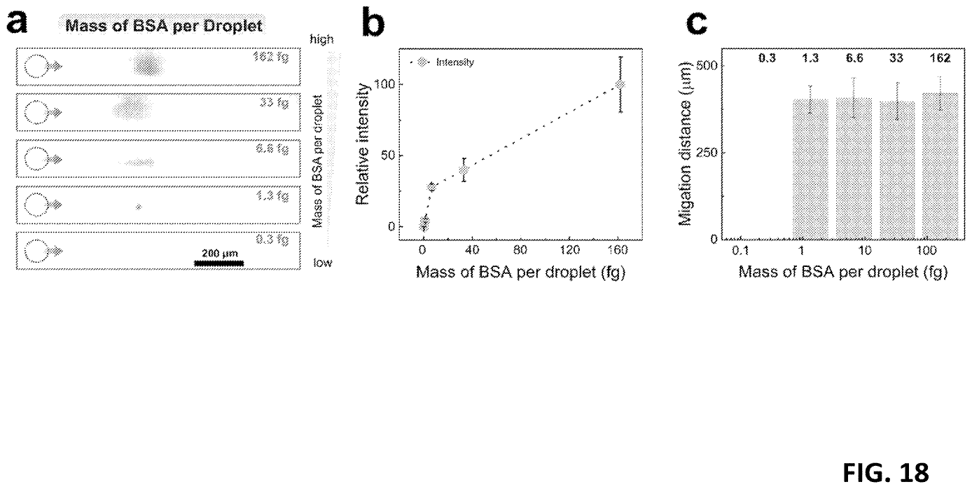

Additionally, the separation performance of BSA (MW: 66 kDa) and IgG (MW: 150 kDa) proteins was investigated at different electrophoresis time (E=40 V/cm). BSA and IgG proteins were diluted to 0.1 mg/mL and encapsulate in in 45 μm droplets containing 0.5% (w/v) SDS. The BSA and IgG can be completely separated after 30 s′ electrophoresis (FIG. 12d). The average migration distances of BSA and IgG are 406.3±16.3 μm and 137.4+20.2 μm, respectively (n=500) with an average separation resolution of 8.1 (n=500, FIG. 4e). As the electrophoresis time increased (45 s, FIG. 12f), the migration distance and separation resolution increased. The average migration distances of BSA and IgG are 627.3±34.1 μm and 230.4±16.8 μm, respectively (n=500) with an average separation resolution of 6.5 (n=500, FIG. 4g). The difference in separation resolution is mainly caused by the electrolysis which can affect the electrophoretic mobility and change the electric field by producing acid and base ions during electrophoresis.⁵ The electrophoretic mobility of IgG in the 8% T gel is about 1550 μm²/(V·s), while the electrophoretic mobility of BSA is 3683 μm²/(V·s). Further, the lowest detection limit of DropBlot with BSA was investigated. The lowest detection limit is 1.3 fg (~600 molecules). (FIG. 18).

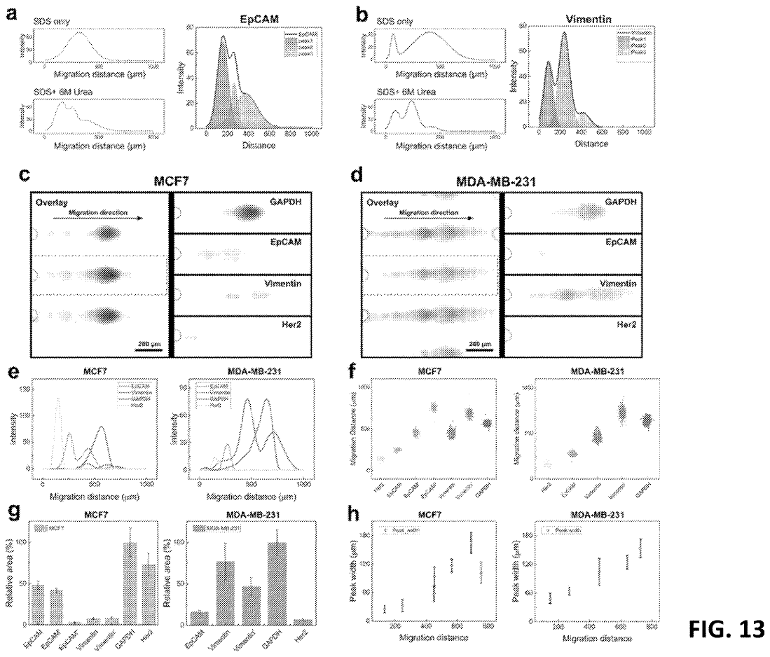

Application of DropBlot to Live Cancer Cells. Using optimized experimental conditions (Diameter of microwell: 50 μm, Diameter of droplet: 45 μm, droplet position: right edge), the DropBlot device was validated with fresh cancer cell lines. A single droplet has a limited amount of SDS to coat proteins in SDS. The 45 μm droplet containing 0.5% (w/v) SDS has about 240 fg SDS while a cell contain about 100 fg proteins. Typically, proteins bind SDS with a constant mass ratio of 1.4 to 1 (SDS: protein)⁶ or 3 to 1 (SDS: protein)⁷. The current droplet (45 μm) and lysis buffer (0.5% (w/v) SDS) combination is thus sufficient to coat all proteins in a single cell. The protein solubility of epithelial cellular adhesion molecule (EpCAM) and intermediate filament protein (Vimentin) was additionally with different lysis buffer. Urea, a strong chaotropic agent, can break hydrogen bonds and unfold hydrophobic protein regions by disrupting hydrophobic interactions.⁸ SDS based lysis buffer supplemented with high concentration of urea (e.g., 6M) can solubilize different proteoforms by reducing detergent micelles and breaking detergent-protein complexes.⁹⁻¹⁰ As is shown in FIG. 13a-b, 0.5% (w/v) SDS (sodium dodecyl sulphate) lysis buffer supplemented with 6M urea can resolve more EpCAM and Vimentin isoforms compared to 0.5% (w/v) SDS only lysis buffer.

The system was applied to immunoblotting analysis of EpCAM, Vimentin, endogenous protein GAPDH and human epidermal growth factor receptor 2 (Her2). DropBlot has successfully resolved proteins (EpCAM, vimentin, HER2, and GAPDH) in human breast cancer cell lines including MCF7 (FIG. 13c) and MDA-MB-231 (FIG. 13d). The protein type was identified and corresponding migration distance from these immunofluorescence images. As depicted in FIG. 13c-e, MCF7 has a high expression of EpCAM and Her2 and low expression of Vimentin, while MDA-MB-231 has a high expression of Vimentin and low expression of EpCAM and Her2. This is consistent with previous research that MCF7 is a epithelial cell line while MDA-MB-231 is a mesenchymal cell line. The epithelial-to-mesenchymal transition (EMT) can change the expression of EpCAM, Vimentin, and Her2.11-12 As is shown in FIG. 13f, the EpCAM peaks are different between MCF7 and MDA-MB-231 cell lines. The MCF7 cells has 3 EpCAM peaks while MDA-MB-231 only has one peak. This is probably due to the different subtypes of EpCAM in these cell lines or different lysis efficiency. The migration distance is proportional to the molecular weight, which also determines diffusion coefficient and peak width (FIG. 13h).

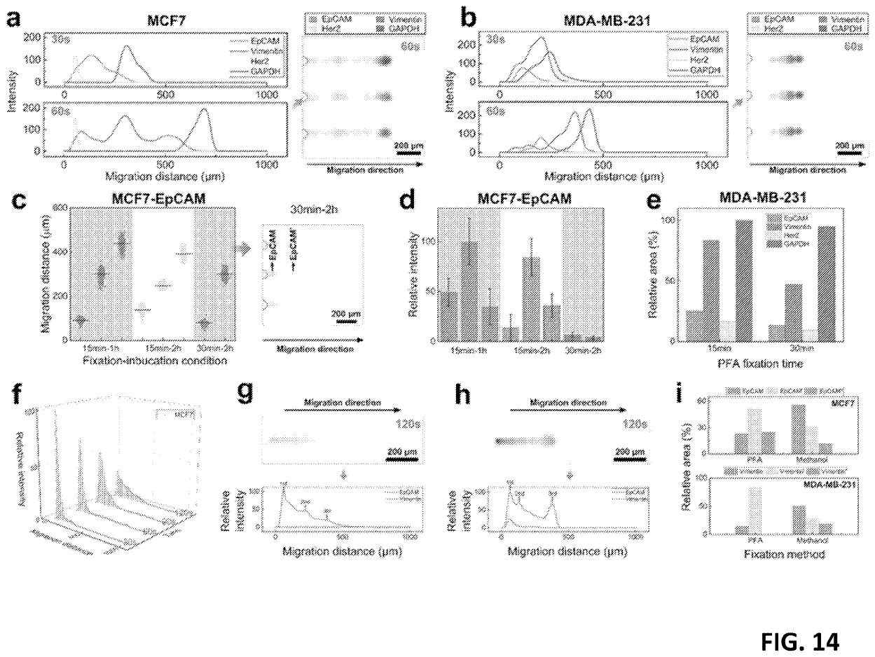

Application of DropBlot to Fixed Cancer Cell Lines. Two types of fixative was used: paraformaldehyde (PFA) and methanol. To retrieve antigens from the fixed cells, the cells were incubated under 98° C. for 60-120 min. As is shown in FIG. 14a-b DropBlot can identify the proteins in PFA-fixed cancer cell lines (MCF7 and MDA-Mb-231, fixation time: 15 min). Proteins moved slower in fixed cancer cells than that in live cancer cells. It took about 60s to have proteins in fixed cells migrate similar distance. The slow migration speed was due to the incomplete protein recovery. The PFA fixation time and antigen-retrieval conditions determine the protein migration distance and number of protein signals. As is shown in FIG. 146c-e, increasing the incubation time (2h) can solubilize more proteins. However, increasing the fixation time (30 min) dramatically reduce the peak intensity and number. Long-time PFA fixation can increase the crosslink strength between protein and protein or protein and lipid. Methanol fixation can promote the protein aggregation without form crosslinks. Using same lysis buffer with FPA experiment, EpCAM in methanol-fixed MCF7 cells moved even much slower (FIG. 6f). The protein signals were much higher near the microwells (FIG. 14g-h), representing high amount of protein aggregates. The integrated peak area of EpCAM and Vimentin in MCF7 and MDA-MB-231 were compared and further proved that methanol fixation has a significant amount insoluble protein (FIG. 14i).

### Materials and Methods

Modeling and Simulation. Electric field and protein electromigration were simulated using finite-element modeling of electric current and diluted species transport with COMSOL Multiphysics (version 5.5, COMSOL Inc., Sweden). The simulation geometry for the DropBlot electrophoresis are presented in the top view shown in FIG. 12a. Diffusion coefficients in free solution and oil layer were calculated based on the Stokes-Einstein equation³, while the diffusion coefficient in gel layer was estimated based on the published method¹³. A temperature of 23° C. was used in all simulations. The initial concentration in the droplet region was 2 μM, while the initial concentration elsewhere in the system was 0 μM. The model was meshed with a Free Triangle mesh, and a user-controlled override with a maximum element size of 2 μm and a minimum element size of 0.01 μm in the oil layer regions to provide a large-enough mesh density in the narrow region.

Fabrication of Droplet Generation Chips. Single emulsion generation chips were fabricated using standard soft lithographic techniques. SU-8 3050 (Kayaku Advanced Materials, Westborough, MA) was used to fabricate masters with a height of 60 μm. PDMS was prepared with Sylgard 184 Silicone Elastomer Kit (Ellsworth Adhesives, Germantown, WI) and mixed with a ratio of 10:1. After curing (70° C., 3 hours), the PDMS device was further baked for 48 hours at 80° C. to recover the hydrophobicity.

Polyacrylamide Gel fabrication. Wafer microfabrication and silanization are described in our previous work¹⁴. Microwell arrays in this design has a diameter of 50 μm and a thickness of 60 μm. To prepare an 8% T polyacrylamide gel, the gel precursor solution was mixed with 30% (wt/wt) Acrylamide/bis-acrylamide (Sigma-Aldrich, St. Louis, MO), N-(3-((3-benzoylphenyl) formamido) propyl) methacrylamide (BPMA, PharmAgra Labs, Brevard, NC), 10X tris-glycine buffer (Sigma-Aldrich, St. Louis, MO), and ddH₂O (Sigma-Aldrich, St. Louis, MO). Gels were chemically polymerized for 20 min with 0.08% (w/v) ammonium persulfate (APS, Sigma-Aldrich, St. Louis, MO) and 0.08% (v/v) TEMED (Sigma-Aldrich, St. Louis, MO). After polymerization, gels were collected with a razor blade, released from the wafer, and stored in the Milli-Q water.

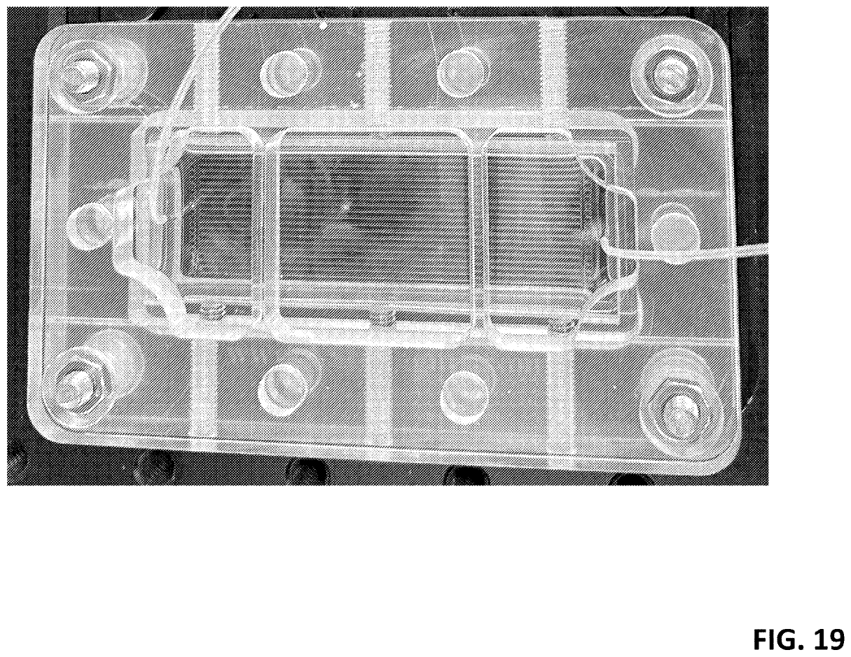

Fluidics Assembly and Generation of W/O droplet. Harvested cells or purified proteins were resuspended in PBS and injected into the cell inlet. Cell suspension and lysis buffer were mixed prior to the emulsion region. 1-2% (v/v) Span 80 surfactant (Sigma Aldrich, St. Louis, MO) was spiked into mineral oil (Sigma Aldrich, St. Louis, MO) and used as the carrier solution. Flow rates of the substrate and carrier solution were actively controlled by a syringe pump (Chemyx, Stafford, TX). The flow rates of each solution were adjusted to generate droplets of different sizes. Droplets could be collected in a 1.5 mL Eppendorf tube or be directly loaded onto PA-gel with customized PDMS channels and holder (FIG. 19).

Protein Diffusion Assay. Proteins of different molecular weights were diluted to 5 M with PBS. In this assay, Alexa-Fluor 488 labeled immunoglobulin (IgG, Thermo Fisher Scientific, Waltham, MA), Alexa-Fluor 555 labeled Bovine Serum Albumin (BSA, Thermo Fisher Scientific, Waltham, MA), Alexa-Fluor labeled Ovalbumin 647 (OVA, Thermo Fisher Scientific, Waltham, MA), rTurboGFP (GFP, Evrogen, Russia) were used. Proteins were encapsulated into 50 μm droplets and observed under an inverted microscope (Olympus) for 180 min.

Protein Electrophoresis using DropBlot. Protein-loaded droplets were settled down on the PA-gel, which was placed in a customized electrophoresis chamber (width: 5 cm). 12.5 mL running buffer, containing 1X Tris-Glycine and 0.5% (v/v) Sodium Dodecyl Sulfate (SDS, Sigma Aldrich, St. Louis, MO), was poured onto the chamber. A constant voltage of 200-300 V (the target voltage for an electric field of 40-60 V/cm) was supplied using a DC power supply (Bio-Rad PowerPac Basic, Hercules, CA). Immediately After electrophoresis, the power was shut off and migrated proteins were photo-captured by 45 s UV exposure with a Hamamatsu Lightingcure LC5 UV source (HAMAMATSU PHOTONICS, Japan). The slides were then rinsed with Milli-Q water and imaged with an inverted microscope and Genepix Microarray Scanner (Molecular Devices, San Jose, CA).

Cell Culture. Human breast cancer cell lines, including MCF7, MCF7/GFP, and MDA-MB-231/GFP (ATCC, Manassas, VA), were used in this study. Cells were cultured in DMEM medium (Thermo Fisher Scientific, Waltham, MA) supplemented with 10% (v/v) fetal bovine serum (GeminiBio, West Sacramento, CA), 1% (v/v) penicillin/streptomycin solution (Thermo Fisher Scientific, Waltham, MA), and 0.1 mM non-essential amino acid solution (Thermo Fisher Scientific, Waltham, MA). The cells were cultured in the incubator (37° C., 5% CO2) and were released through incubation with 0.05% trypsin-EDTA solution (Thermo Fisher Scientific, Waltham, MA). The concentration of harvested cells was measured with a hemocytometer (Hausser Scientific, Horsham, PA) and resuspended with Phosphate Buffered Saline (PBS, Thermo Fisher Scientific, Waltham, MA) to a specific concentration.

Application of DropBlot on Live Cells. Cancer cell lines, MCF7 and MDA-MB-231, were resuspended with PBS to a concentration of 5e6 cells/mL. The Lysis buffer used for live cell was 2X Tris-Glycine buffer (Sigma Aldrich, St. Louis, MO) supplemented with 1% SDS and 12 M urea (Sigma Aldrich, St. Louis, MO). Flow rates of cell solution, lysis buffer, and carrier layer (oil) were 1, 1, 12 μL/min to generate droplets with a diameter of ~45 μm. The final concentration of lysis buffer was 1X Tris-Glycine buffer supplemented with 0.5% SDS and 6 M urea. Cell-laden droplets were then loaded on the 8% T polyacrylamide gel. The PDMS slab was then relocated to cover the droplet-loaden microwells and the extra mineral oil was flushed out with running buffer (1X Tris-Glycine supplemented with 1% SDS). Cells should be lysed for at least 10 min in the droplet at room temperature. 12.5 mL of running buffer was poured into the chamber and the separation was initiated by supplying 300V constant voltage for an average electric field of 40-60 V/cm across the gel for 30s. After electrophoresis, the proteins were photo-captured by 45 s′ UV exposure. The gel was then rinsed briefly with deionized water and stored in tris-buffered saline with Tween 20 (TBST, Cell Signaling Technology, Danvers, MA) overnight to remove excess oil and residual lysis buffer.

Application of DropBlot on PFA-fixed Cells. Cancer cell lines, MCF7 and MDA-MB-231, were fixed with 4% Paraformaldehyde (PFA, Alfa Aesar, Haverhill, MA) for 15-30 min at room temperature. After fixation, cells were washed three times with PBS to remove excess PFA and resuspended with PBS to achieve a concentration of ~5e6 cells/mL. The Lysis buffer used for live cell was 2X Tris-Glycine buffer supplemented with 2% SDS and 12 M urea Flow rates of cell solution, lysis buffer, and carrier layer (oil) were 0.5, 0.5, 5 L/min to generate droplets with a diameter of ~45 μm. The final concentration of lysis buffer was 1X Tris-Glycine buffer supplemented with 1% SDS and 6 M urea. The droplets were collected and incubated in a 1.5 mL Eppendorf tube at 98° C. for 1-2 hours, and then loaded onto 8% T polyacrylamide gel. 12.5 mL of running buffer was poured into the chamber and the separation was initiated by supplying 300V constant voltage for an average electric field of 40-60 V/cm across the gel for 30-60s. After electrophoresis, the proteins were photo-captured by 45 s′ UV exposure. The gel was then rinsed briefly with deionized water and stored in tris-buffered saline with Tween 20 (TBST).

Application of DropBlot on Methanol-fixed Cells. Cancer cell lines, MCF7 and MDA-MB-231, were fixed with ice-cold methanol for 15 min at −20° C. After fixation, cells were washed three times with PBS to remove excess methanol and resuspended with PBS to achieve a concentration of ~5e6 cells/mL. The Lysis buffer used for live cell was 2X Tris-Glycine buffer supplemented with 2% SDS and 12 M urea Flow rates of cell solution, lysis buffer, and carrier layer (oil) were 0.5, 0.5, 5 L/min to generate droplets with a diameter of ~45 μm. The final concentration of lysis buffer was 1X Tris-Glycine buffer supplemented with 1% SDS and 6 M urea. The droplets were collected and incubated in a 1.5 mL Eppendorf tube at 98° C. for 1 hour, and then loaded onto 8% T polyacrylamide gel. 12.5 mL of running buffer was poured into the chamber and the separation was initiated by supplying 300V constant voltage for an average electric field of 40-60 V/cm across the gel for 30-120s. After electrophoresis, the proteins were photo-captured by 45 s′ UV exposure. The gel was then rinsed briefly with deionized water and stored in tris-buffered saline with Tween 20 (TBST).

### Application of DropBlot on Fixed Clinical Samples:

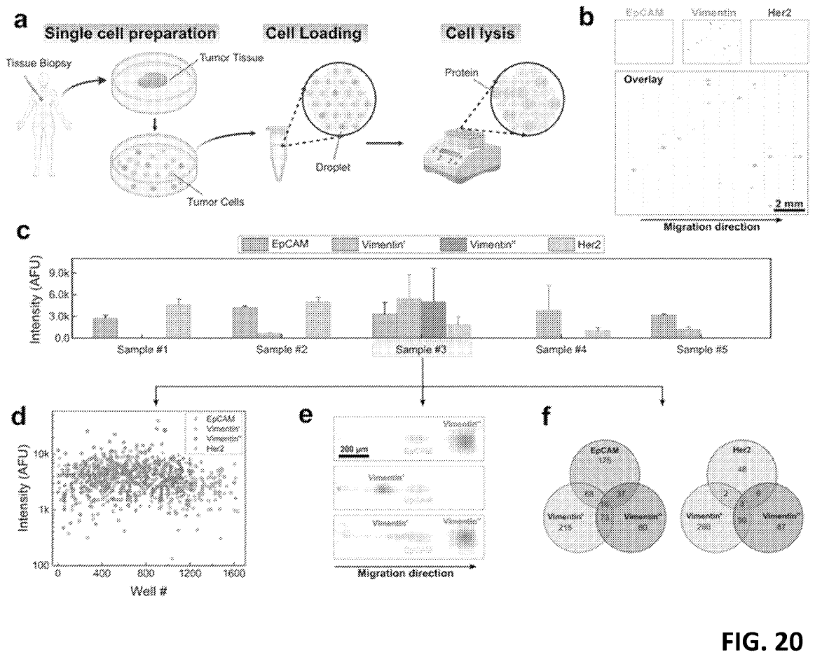

Evaluate the performance of DropBlot in resolving proteoforms in fixed clinical samples, a study of samples collected from 6 patients exhibiting breast cancer was conducted. DropBlot device was used to process cell/tissue samples from the patients, who were recruited and gave informed consent under both Stanford and Berkeley Institutional Review Board (IRB) permission. Protein targets corresponding to epithelial-to-mesenchymal transition (EMT) and tumor cell growth were chosen because the tumor cells with the mesenchymal phenotype and high expression of Her2 tend to be more aggressive¹⁵⁻¹⁸. Samples used in this assay were collected more than 6 years ago and were stored in −80° C. freezer. Prior to loading into the droplets, the tissue samples need to be processed to achieve single-cell suspension. These fresh samples were fixed with 4% PFA for 15 min to prevent the degradation of protein molecules (FIG. 20a). Cell suspension or tissue samples was processed by the DropBlot device. Protein and proteoforms in single tumor cells were analyzed through immunofluorescence staining with an epithelial marker (EpCAM), mesenchymal marker (Vimentin), and human epidermal growth receptor 2 (Her2). An example of separation gel is shown in FIG. 20b. It is noted that expression levels of EpCAM, Vimentin, and Her2 are heterogeneous among breast tumor cells. By investigating the expression level of these proteins, DropBlot could provide information regarding the EMT status, EpCAM/Vimentin proteoforms, and Her2 expression level, and thus facilitating the prediction of patient's risk of developing cancer metastasis and promoting the development of precision therapy in human disease.

The patient information, including ER-α, PR, and Her2 status, is summarized in Table 1 and the corresponding protein profiles (mean intensity, is shown in FIG. 20c. The mean intensity (n≈1000) of proteins in different patients is heterogeneous, and there are two Vimentin proteoforms (Vimentin′, Vimentin ″) founded in the sample. For example, patient #2 has a high expression of EpCAM and Her2, low expression of Vimentin′, and no expression of Vimentin″, while patient #3 has high expression of Vimentin′, low expression of Her2, and no expression of EpCAM and Vimentin ″. It is interesting to see the different expression levels of Vimentin proteoforms. The 55 kDA intermediate filament is the most prevalent and commonly found form of vimentin¹⁹, truncated Vimentin proteoforms with reduced molecular weight can be generated due to cancer metastasis²⁰⁻²². Patient #3 has two types of Vimentin proteoforms, which may be correlated to increased cancer invasiveness. Additionally, the expression of Her2 expression in Her2-Negative sample (#1 and #3). This is because Her2 can also express in normal cells and the upregulated expression of Her2 indicates Her2-Positive¹⁸.

Her2-positive metastatic breast cancer tends to have a strong response to first-line treatment with trastuzumab²³, investigating the expression of Her2 and metastatic markers (EpCAM and Vimentin) can help predict the drug resistance and treatment outcome. The protein profiles of individual tumor cells from patient #3 were assayed, which is invasive ductal breast tumor (FIG. 20d). The expression of EpCAM, Vimentin′, Vimentin″, and Her2 expression is heterogeneous among individual tumor cells. As is shown in FIG. 20e, the cell can be EpCAM+/Vimentin′−/Vimentin″+, EpCAM+/Vimentin′+/Vimentin″−, or EpCAM+/Vimentin′+/Vimentin″+. There are about 9.3% of cells expressing EpCAM+/Vimentin′−/Vimentin″+, 5.7% of cells expressing EpCAM+/Vimentin′+/Vimentin″−, and 2.5% Of cells expressing EpCAM+/Vimentin′+/Vimentin″+. Furthermore, the coexpression of Her2 and Vimentin Proteoforms were summarized, and it was found that there about 0.4% of cells expressing Her2+/Vimentin′+/Vimentin″−, 1.2% of cells expressing Her2+/Vimentin′−/Vimentin″+, and 0.6% of cells expressing Her2+/Vimentin′+/Vimentin″+.

TABLE 1
Clinical samples tested with DropBlot

| | | ER-α | PR, HER2 | | | |
|---|---|---|---|---|---|---|
| # | Patient | Status | Status | Cell Status | Type | Signal |
| 1 | 041318 | ER-α³⁺ | PR+, | Suspension, | Invasive ductal breast | Yes |
| | | | HER2⁻ | Fresh | tumor, PT2pN0 | (EpCAM) |
| 2 | 121715 | ER-α¹⁺ | PR−, | Suspension, | Lymph node infiltrated | Yes |
| | | | HER2⁺ | Fresh | breast tumor | |
| 3 | 041318 | ER-α³⁺ | PR+, | Tissue, Fresh | Invasive ductal breast | Yes |
| | | | HER2⁻ | | tumor | |
| 4 | 102816 | ER-α¹⁺ | PR+, | Tissue, Fresh | Triple Positive breast | Yes |
| | | | HER2⁺ | | tumor | |
| 5 | 040615 | — | — | Tissue, Fresh | Cureline Bca Fresh | Yes |
| | | | | | Tissue | |

DropBlot analysis of fresh (not fixed) cancer cells. Next, the preparation and analysis of unfixed cells was scrutinized, testing the sample preparation and integration functions of DropBlot. To assess the relationship between antigen-retrieval buffer formulation (including SDS) and the immunoreactivity of any retrieved antigen, fresh cells were scrutinized from well-studied cultured cell lines and the protein targets epithelial cellular adhesion molecule (EpCAM, 35-40 kDa) and intermediate filament protein (VIM, 57 kDa). We anticipate the chemical state of each originating cell will impact the degree of antigen retrieval for each protein target. Antigen retrieval means, most obviously, the amount of antigen material recovered from a chemically fixed cell. Importantly, and more subtly, antigen retrieval also depends on the degree of recovery of immunoreactivity, which in turn depends on the physicochemical properties of each retrieved antigen species. In this latter aspect, fixation-induced alterations in protein physicochemical properties are expected to inherently affect electromigration. Consequently, protein-target identity in the simplest to the most complex matrix conditions were established using a modified ‘spiked recovery’ immunoassay development process to account—as much as possible—for matrix effects anticipated in human-derived and chemically fixed cell specimens²⁴. Here, that means starting with target antigen measurement from a clear buffer, progressing next to antigen measurement from fresh cell lines, then retrieval from fixed cells from the same cell lines, and finally move to considering retrieval of a panel of protein targets in fixed patient-derived tumor specimens. It is assumed that SDS binds proteins with a constant mass ratio (i.e., 1.4 to 1 (SDS: protein)²⁵ or 3 to 1 (SDS: protein)²⁶), each 45-μm diameter droplet contains ~240 fg of SDS, and that each mammalian cell contains ~100 fg of protein. The selected droplet volume (Ø=45 μm, volume=382 pL) and the selected antigen-retrieval buffer (0.5% (w/v) SDS) provide a mass of SDS sufficient to coat all protein molecules from each single cell. As shown in Error! Reference source not found., solubilization, electrotransfer, and PAGE analysis were demonstrated using this combination. Immunoreactivity was sufficiently recovered for each protein target, based on successful endpoint immunoblotting.

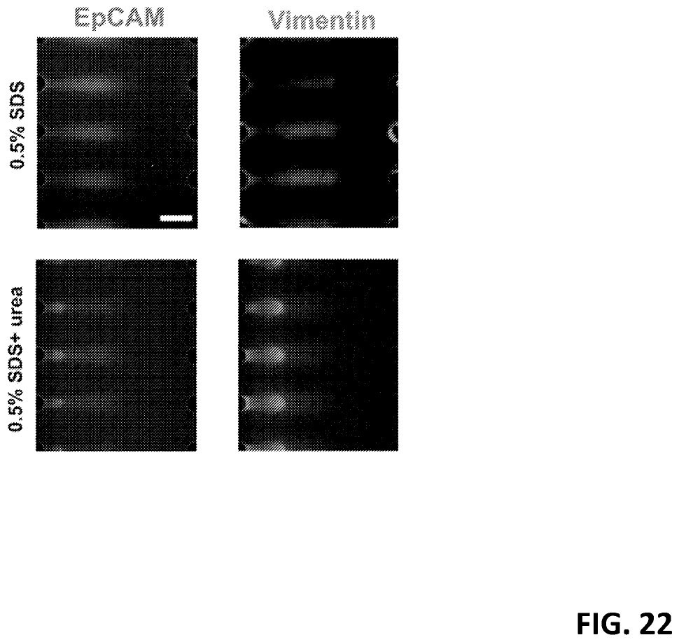

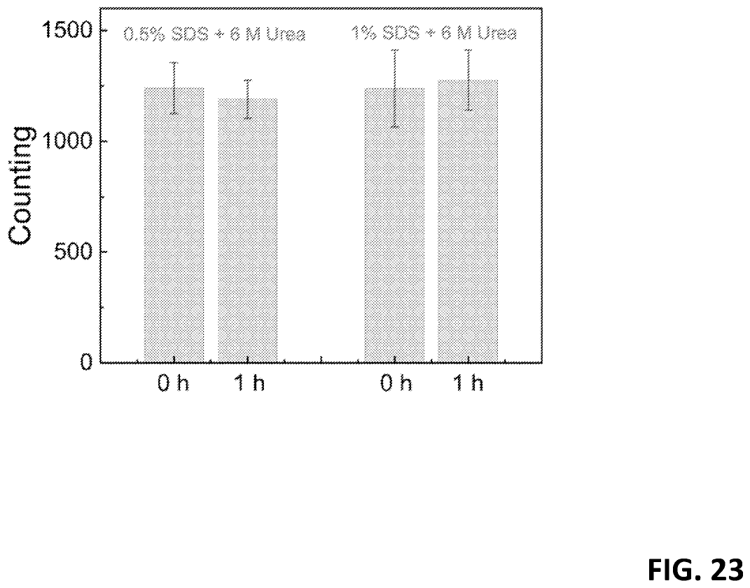

Expanding the repertoire of cell preparation conditions accessible with DropBlot, we explored the possibility of including 6 M urea in the 0.5% (w/v) SDS antigen-retrieval buffer (Error! Reference source not found.a-b, FIG. 22). Urea, a strong chaotropic agent, can break hydrogen bonds and unfold hydrophobic protein regions by disrupting hydrophobic interactions²⁷. SDS-based antigen-retrieval buffer supplemented with a high concentration of urea (e.g., 6 M) can solubilize a variety of proteoforms by reducing detergent micelles and breaking detergent-protein complexes^{28,29}. An antigen-retrieval buffer (0.5% (w/v) SDS) supplemented with 6 M urea resolved additional EpCAM and VIM proteoforms during PAGE of single unfixed cells, with no obvious detrimental effect of the urea supplement on droplet stability (FIG. 23).

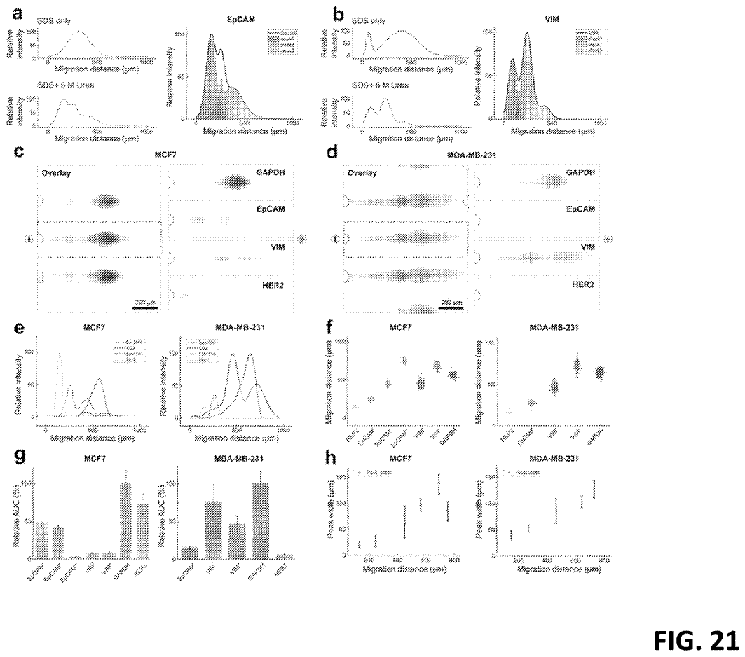

After establishing the DropBlot system as suitable for the analysis of protein targets from unfixed single cells, the multiplexed cancer-protein panel was analyzed by immunoblotting the targets EpCAM, VIM, endogenous protein GAPDH, and human epidermal growth factor receptor 2 (HER2). In both a human breast epithelial line (MCF7) and a triple-negative breast cancer line (MDA-MB-231), DropBlot successfully completed unfixed cell and protein sample preparation, then supported immunoblotting of the four targets as shown in FIG. 21c-d. No cross-reactivity was observed between the antibody probes utilized and the four targets from the cancer-protein panel.

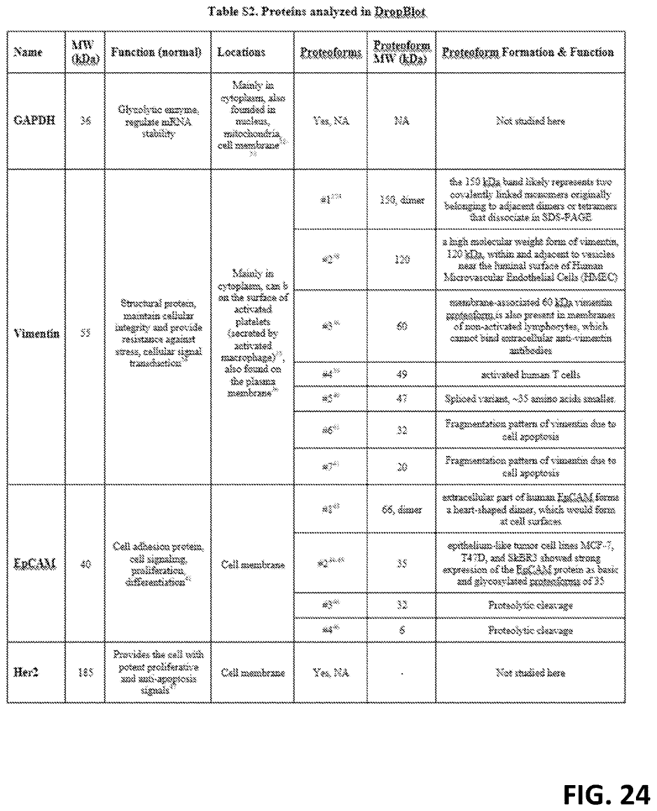

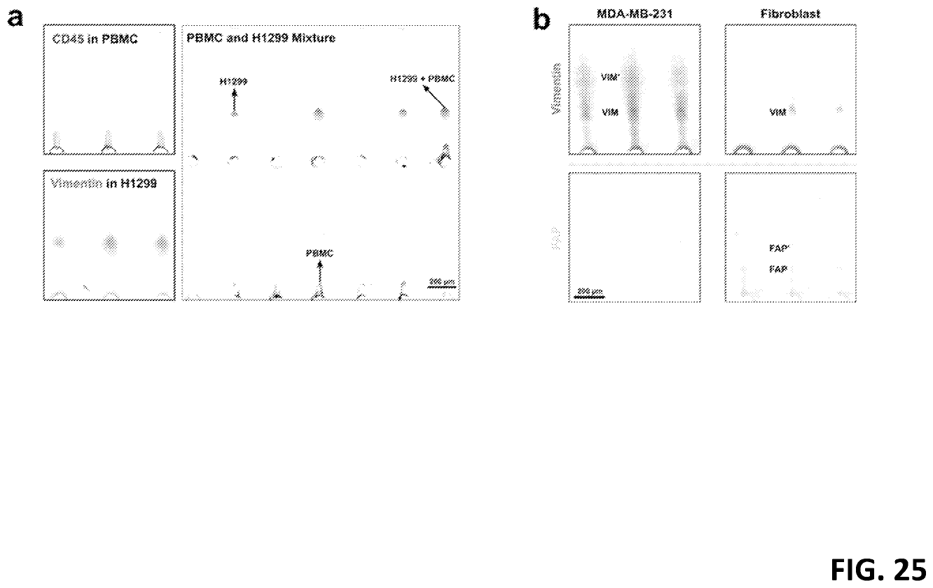

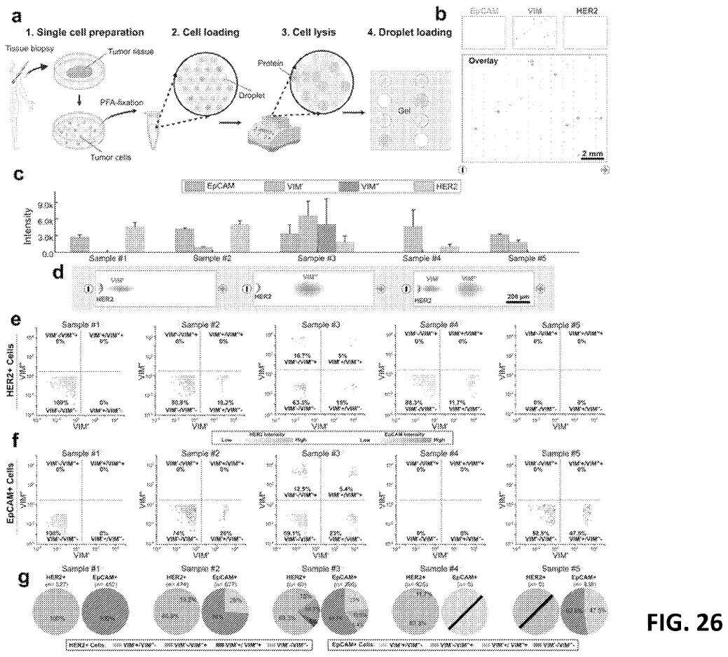

Consistent with previous research, the epithelial cell line, MCF7, had a high expression of EpCAM and HER2 and low expression of VIM, relative to a mesenchymal cell line (MDA-MB-231), which had a high expression of VIM and low expression of EpCAM and HER2. The epithelial-to-mesenchymal transition (EMT) can alter the expression of EpCAM, VIM, and HER230, 31. Three resolved EpCAM peaks in the lysate of single MCF7 cells were also observed, while MDA-MB-231 cells exhibited one detectable peak (FIG. 21e-h). We attribute the cell-line-dependent EpCAM expression to different proteoforms of EpCAM (FIG. 24). In the intricate interplay of cellular distinctions, DropBlot emerges as a discerning tool, capable of delineating between cell types based on their unique fingerprint proteins and proteoforms (FIG. 25). Notably, the H1299 lung cancer cell line is demarcated from PBMCs by the distinct presence of CD45 and VIM⁴⁸, while discriminating between MDA-MB-231 cells and stromal fibroblasts can rely on nuanced variations in VIM and fibroblast activation protein (FAP) expression levels and proteoforms. Although FAP is also expressed on MDA-MB-231, the expression level and types of proteoforms vary⁴⁹.

DropBlot analysis of fixed clinical specimens reports co-expression of two VIM proteoforms in a rare sub-population of HER2+tumor cells. As a first demonstration of the DropBlot assay after assay development and validation, analysis of a well-understood protein panel in chemically fixed human-derived biospecimens was demonstrated. As mentioned, the panel consisted of an epithelial marker (EpCAM), a mesenchymal marker exhibiting proteoforms (VIM′, VIM″), and human epidermal growth receptor 2 (HER2). In particular, HER2 and VIM were chosen because tumor cells with a mesenchymal phenotype and high expression of HER2 tend to be more aggressive^{50, 51}.

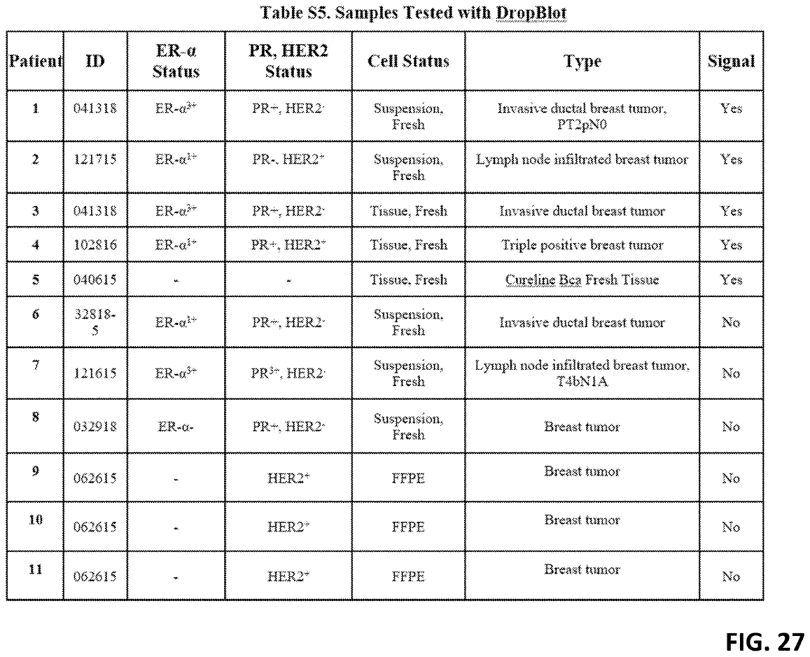

Dropblot was used to scrutinize single, fixed cells dissociated from 11 solid breast tumor specimens. These human-derived tumor tissues were archived for >6 yrs. stored under −80° C. conditions without chemical fixation. Prior to DropBlot analysis, these patient-derived cells were thawed, tissue was dissociated, and PFA-fixation completed (FIG. 26a, FIG. 27). DropBlot successfully retrieved antigen from 5 of the PFA-fixed cell specimens, as determined by probing for markers of epithelial-to-mesenchymal transition (EMT) and tumor cell growth at the single-cell level (FIG. 26b-c). Having now validated DropBlot on complex human-derived tissue specimens, the sources of VIM-proteoform expression heterogeneity in these specimens was evaluated, at the level of originating cell type with a particular interest in HER2+ cancer cells (FIG. 26d).

To distinguish cancer cells from among the other cell types present (i.e., stromal, immune), DropBlot in a ‘cell gating’ mode was used-analogous to the gating functionality commonly used in flow cytometry. Gating on protein marker expression allows analysis of specific cellular sub-populations in a manner like flow cytometry, with DropBlot enabling analysis of protein proteoforms at the single-cell level, even when antibody probes specific to each proteoform have either poor performance or are nonexistent. This latter functionality is not possible using flow cytometry, or other existing single-cell immunoassays (i.e., immunofluorescence, IHC, mass cytometry).

Employing DropBlot in cell gating mode to hone in on HER2+ cancer cells, we were particularly interested in cancer cells as sources of VIM and VIM-proteoform expression and heterogeneity (FIG. 26e-g). We identified HER2+ cell sub-populations across the five human-derived tumor specimens from which antigen was successfully retrieved after chemical fixation. Attending to Sample #3 in a moment, Samples #1 and #2 were fresh cell suspensions and contained 26.4% and 38.9% HER2+ cells, respectively. While 26.4% of Sample #1 cells and 31.4% of Sample #2 cells co-expressed HER2 and VIM′, there were no cells detectable that expressed the VIM″ proteoform (either alone or with VIM). Samples #4 and #5, which were suspensions of fresh dissociated tissues, showed 46.3% and 0% cells as HER2+, respectively. In Sample #4, 40.8% cells co-expressed HER2+ and VIM+, with VIM expression arising only from the VIM′ proteoform. VIM″ was not detected in Sample #4, either alone or co-expressed with VIM′. Sample #5 reported a cell sub-population of 41.9% of the cells analyzed, which was expressing VIM′ alone and likely stromal in origin.

Returning to Sample #3, the 707 cells analyzed were composed of 8.5% HER2+ cells by DropBlot. DropBlot detected HER2+with no co-expression of VIM in ~5.4% of the cells assayed. Nearly ~2.7% of HER2+ cells expressed one but not the other VIM proteoform (1.3% VIM′ only and 1.4% VIM″ only). Importantly, DropBlot detected a rare cell sub-population of ~0.4% of the analyzed cells that were HER2+ and co-expressed both VIM′ and VIM″ proteoforms.

Importantly, Sample #3 was classified as an invasive ductal carcinoma. Among the HER2+ cell sub-populations surveyed in this pilot study, Sample #3 proved to be the most heterogeneous in VIM proteoform expression. Previous research shows that the mesenchymal phenotype and high expression of HER2 tend to be indicative of a more aggressive phenotype^{50, 51}. Further, previous studies suggest that the most prevalent form of VIM is the 55-kDa intermediate filament⁵², with truncated VIM generated during cancer metastasis⁵³⁻⁵⁵.

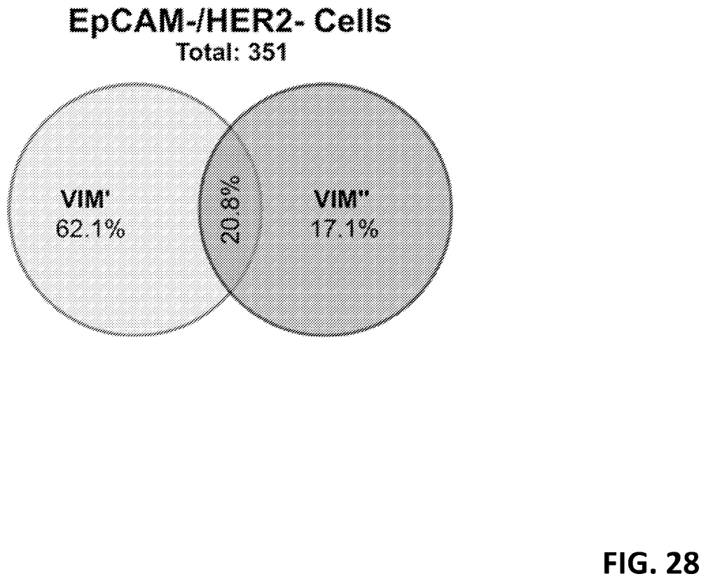

Applying DropBlot and gating on sub-populations of non-cancerous cells, we hypothesized that cells expressing VIM only (either form; but no HER2) may represent stromal cells. Gating on total VIM expression, DropBlot reported ~50% of cells analyzed from Sample #3 expressed VIM with co-expression of no other panel marker for this tumor dissociate. Given the prevalence of the VIM-only expressing cells, stromal cells are the likely originating cell type, as would be expected in a dissociated tumor. In the VIM+stromal cells of Sample #3, 30.8% expressed VIM′, 8.5% expressed VIM″, and 10.3% co-expressed VIM′ and VIM″ (FIG. 28).

In a novel function of the DropBlot assay, the cell population was gated based on expression of just one of the VIM proteoforms. Just ~1.3% of the cells analyzed from Sample #3 co-expressed VIM′ with HER2 and ~9.6% co-expressed VIM′ with EpCAM. Next gating on expression of the second VIM″ proteoform only, DropBlot identified ~1.4% of the cells analyzed from Sample #3 as co-expressing VIM″ with HER2 and ~5.2% co-expressing VIM″ with EpCAM. Roughly 14.9% of the Sample #3 cells analyzed were positive for VIM (VIM′ or VIM″) and EpCAM, with ~2.3% expressing both VIM proteoforms and EpCAM.

Based on these results and demonstrated sufficient performance for PFA- and methanol-fixed cells, additional sample preparation conditions can be evaluated to determine if additional fixation chemistries are compatible with the sample preparation-to-analysis workflow.

Fresh tumor specimens (cell suspension or tissue). Frozen samples were thawed in a water bath at 37° C. for 1 min and mixed with 10 mL of DMEM medium. Samples were centrifuged at 300×g for 5 min to remove the supernatant. For the fresh cell suspension, the samples were fixed with 4% PFA for 15 min and resuspended with PBS to a concentration of 4×10⁶ cells/mL. For the fresh tissue (FIG. 26a), a 1-g tissue specimen was weighed and placed in a petri dish containing 5 mL of 37° C. DMEM medium. Using a scalpel and tweezer, the tissue was coarsely dissected into fragments <0.75 mm in diameter. A tissue suspension was constituted by adding 5 mL of Tumor & Tissue Dissociation Regent (TTDR, BD Bioscience, San Jose, CA), and then incubating the mixture at 37° C. for 30 min with frequent agitation. After incubation, 25 mL of Dulbecco's Phosphate Buffered Saline (DPBS, Thermo Fisher Scientific, Waltham, MA) containing 1% BSA and 2 mM EDTA (Thermo Fisher Scientific, Waltham, MA) was added. Large tissue/cell clusters were removed with a 70-μm cell strainer and then centrifuged at 300×g for 5 min to remove the supernatant. A cell pellet formed and was resuspended in 2 mL of 1× lysis buffer (Tonbo Biosciences, San Diego, CA) and incubated at room temperature for 15 min. A 40 mL aliquot of DPBS containing 1% BSA and 2 mM EDTA was then added to the mixture. After removing the supernatant, the cells were fixed following the fixation protocols described elsewhere. Cells were counted using a hemocytometer and resuspended to 4×10⁶ cells/mL with PBS.

Formalin-fixed, paraffin-embedded (FFPE) tumor specimens. Frozen tissue samples were thawed in a water bath at 60° C. for 2 hr, and bathed in 10 mL xylene (Sigma Aldrich, St. Louis, MO) for 5 min (twice). The samples were rehydrated with 96% ethanol, 90% ethanol, 70% ethanol, 50% ethanol, and PBS for 5 min, then washed twice. The cells were fixed following the fixation protocols described elsewhere and resuspended to 4×10⁶ cells/mL with PBS.

Immunoprobing and fluorescence imaging for the cell analysis step. The primary antibody immunoprobing solution was prepared by diluting stock solutions of primary antibodies in 2% (w/v) BSA/TBST solution to achieve an antibody concentration of 0.05 μg/μL (single antibody). Primary antibodies used were EpCAM, VIM, HER2, and GAPDH (Abcam, Cambridge, United Kingdom). The single-cell western blotting device (gel slide) was treated with 80 μL of primary antibody immunoprobing solution and incubated at room temperature for 2 hr. After incubation, each single-cell western blotting device was washed twice with TBST buffer for 1 hour. The secondary antibody immunoprobing solution was prepared by diluting stock solutions of primary antibodies in 2% (w/v) BSA/TBST solution to achieve a concentration of 0.05 μg/μL (single antibody). The single-cell western blotting device was incubated with 80 μL of secondary antibody immunoprobing solution at room temperature for 2 hr. After incubation, the single-cell western blotting device was washed twice with TBST buffer for 1 hr. Before fluorescence imaging, the single-cell western blotting device was washed 3× with DI water to remove excess salts, and dried with nitrogen gun. The single-cell western blotting device was imaged with a Genepix Microarray Scanner. Images were analyzed using custom analysis scripts in MATLAB (MathWorks, Natick, MA). Two to three protein targets were analyzed concurrently. Antigen-target multiplexing utilized an established stripping and re-probing method⁵⁵.

### References Cited in Example 1, Above

1. Ferguson, K. A., Starch-Gel Electrophoresis—Application to the Classification of Pituitary Proteins and Polypeptides. Metabolism 1964, 13, SUPPL: 985-1002.
2. Vlassakis, J.; Herr, A. E., Joule Heating-Induced Dispersion in Open Microfluidic Electrophoretic Cytometry. Anal Chem 2017, 89 (23), 12787-12796.
3. Miller, C. C., The Stokes Einstein Law for Diffusion in Solution. P R Soc Lond a-Conta 1924, 106 (740), 724-749.
4. Vlassakis, J.; Herr, A. E., Joule Heating-Induced Dispersion in Open Microfluidic Electrophoretic Cytometry. Anal Chem 2017, 89 (23), 12787-12796.
5. Macounova, K.; Cabrera, C. R.; Holl, M. R.; Yager, P., Generation of Natural Ph Gradients in Microfluidic Channels for Use in Isoelectric Focusing. Analytical Chemistry 2000, 72 (16), 3745-3751.
6. Reynolds, J. A.; Tanford, C., Binding of Dodecyl Sulfate to Proteins at High Binding Ratios. Possible Implications for the State of Proteins in Biological Membranes. Proceedings of the National Academy of Sciences 1970, 66 (3), 1002-1007.
7. Hames, B. D., Gel Electrophoresis of Proteins: A Practical Approach. OUP Oxford: 1998; Vol. 197.
8. Rabilloud, T., Solubilization of Proteins for Electrophoretic Analyses. Electrophoresis 1996, 17 (5), 813-29.
9. Wisniewski, J. R.; Zougman, A.; Nagaraj, N.; Mann, M., Universal Sample Preparation Method for Proteome Analysis. Nat Methods 2009, 6 (5), 359-62.
10. Glatter, T.; Ahrne, E.; Schmidt, A., Correction to “Comparison of Different Sample Preparation Protocols Reveals Lysis Buffer-Specific Extraction Biases in Gram-Negative Bacteria and Human Cells”. J Proteome Res 2016, 15 (2), 679.
11. Kalluri, R.; Weinberg, R. A., The Basics of Epithelial-Mesenchymal Transition. J Clin Invest 2009, 119 (6), 1420-8.
12. Carpenter, R. L.; Paw, I.; Dewhirst, M. W.; Lo, H.-W., Akt Phosphorylates and Activates Hsf-1 Independent of Heat Shock, Leading to Slug Overexpression and Epithelial—Mesenchymal Transition (Emt) of Her2-Overexpressing Breast Cancer Cells. Oncogene 2015, 34 (5), 546-557.
13. Park, I. H.; Johnson, C. S.; Gabriel, D. A., Probe Diffusion in Polyacrylamide Gels as Observed by Means of Holographic Relaxation Methods—Search for a Universal Equation. Macromolecules 1990, 23 (5), 1548-1553.
14. Rosas-Canyelles, E.; Modzelewski, A. J.; Geldert, A.; He, L.; Herr, A. E., Multimodal Detection of Protein Isoforms and Nucleic Acids from Mouse Pre-Implantation Embryos. Nature protocols 2021, 16 (2), 1062-1088.
15. Lambert, A. W.; Pattabiraman, D. R.; Weinberg, R. A., Emerging Biological Principles of Metastasis. Cell 2017, 168 (4), 670-691.
16. Kim, M. S.; Jeong, J.; Seo, J.; Kim, H. S.; Kim, S. J.; Jin, W., Dysregulated Jak2 Expression by Trkc Promotes Metastasis Potential, and Emt Program of Metastatic Breast Cancer (Vol 6, 33899, 2016). Sci Rep-Uk 2020, 10 (1).
17. Nieto, M. A.; Huang, R. Y. J.; Jackson, R. A.; Thiery, J. P., Emt: 2016. Cell 2016, 166 (1), 21-45.
18. Loibl, S.; Gianni, L., Her2-Positive Breast Cancer. The Lancet 2017, 389 (10087), 2415-2429.
19. Rose, M. L., Role of Anti-Vimentin Antibodies in Allograft Rejection. Hum Immunol 2013, 74 (11), 1459-1462.
20. Whipple, R. A.; Balzer, E. M.; Cho, E. H.; Matrone, M. A.; Yoon, J. R.; Martin, S. S., Vimentin Filaments Support Extension of Tubulin-Based Microtentacles in Detached Breast Tumor Cells. Cancer Research 2008, 68 (14), 5678-5688.
21. Zhang, H.; Wu, X. S.; Xiao, Y. Z.; Wu, L. Q.; Peng, Y.; Tang, W. M.; Liu, G. N.; Sun, Y.; Wang, J.; Zhu, H. Q.; Liu, M. W.; Zhang, W. J.; Dai, W. Y.; Jiang, P.; Li, A. M.; Li, G. X.; Xiang, L.; Liu, S. D.; Wang, J. D., Coexpression of Foxk1 and Vimentin Promotes Emt, Migration, and Invasion in Gastric Cancer Cells. J Mol Med 2019, 97 (2), 163-176.
22. Makise, M.; Nakamura, H.; Kuniyasu, A., The Role of Vimentin in the Tumor Marker Nup88-Dependent Multinucleated Phenotype. Bmc Cancer 2018, 18.
23. Slamon, D. J.; Leyland-Jones, B.; Shak, S.; Fuchs, H.; Paton, V.; Bajamonde, A.; Fleming, T.; Eiermann, W.; Wolter, J.; Pegram, M.; Baselga, J.; Norton, L., Use of Chemotherapy Plus a Monoclonal Antibody against Her2 for Metastatic Breast Cancer That Overexpresses Her2. New Engl J Med 2001, 344 (11), 783-792.
24. Cox, K. L.; Devanarayan, V.; Kriauciunas, A.; Manetta, J.; Montrose, C.; Sittampalam, S. Immunoassay methods. Assay Guidance Manual [Internet] 2019.
25. Reynolds, J. A.; Tanford, C. Binding of dodecyl sulfate to proteins at high binding ratios. Possible implications for the state of proteins in biological membranes. Proceedings of the National Academy of Sciences 1970, 66 (3), 1002-1007.
26. Hames, B. D. Gel electrophoresis of proteins: a practical approach; OUP Oxford, 1998.
27. Rabilloud, T. Solubilization of proteins for electrophoretic analyses. Electrophoresis 1996, 17 (5), 813-829. DOI: 10.1002/elps.1150170503.
28. Wisniewski, J. R.; Zougman, A.; Nagaraj, N.; Mann, M. Universal sample preparation method for proteome analysis. Nat Methods 2009, 6 (5), 359-362. DOI: 10.1038/nmeth. 1322.
29. Glatter, T.; Ahrne, E.; Schmidt, A. Correction to “Comparison of Different Sample Preparation Protocols Reveals Lysis Buffer-Specific Extraction Biases in Gram-Negative Bacteria and Human Cells”. J Proteome Res 2016, 15 (2), 679. DOI: 10.1021/acs.jproteome.6b00012.
30. Kalluri, R.; Weinberg, R. A. The basics of epithelial-mesenchymal transition. J Clin Invest 2009, 119 (6), 1420-1428. DOI: 10.1172/JCI39104.
31. Carpenter, R. L.; Paw, I.; Dewhirst, M. W.; Lo, H.-W. Akt phosphorylates and activates HSF-1 independent of heat shock, leading to Slug overexpression and epithelial-mesenchymal transition (EMT) of HER2-overexpressing breast cancer cells. Oncogene 2015, 34 (5), 546-557.
32. Nicholls, C.; Li, H.; Liu, J. P., Gapdh: A Common Enzyme with Uncommon Functions. Clin Exp Pharmacol Physiol 2012, 39 (8), 674-9.
33. Tristan, C.; Shahani, N.; Sedlak, T. W.; Sawa, A., The Diverse Functions of Gapdh:
Views from Different Subcellular Compartments. Cell Signal 2011, 23 (2), 317-23.
334. Ivaska, J.; Pallari, H. M.; Nevo, J.; Eriksson, J. E., Novel Functions of Vimentin in Cell Adhesion, Migration, and Signaling. Exp Cell Res 2007, 313 (10), 2050-62.
35. Mor-Vaknin, N.; Punturieri, A.; Sitwala, K.; Markovitz, D. M., Vimentin Is Secreted by Activated Macrophages. Nat Cell Biol 2003, 5 (1), 59-63.
36. Bilalic, S.; Michlmayr, A.; Gruber, V.; Buchberger, E.; Burghuber, C.; Bohmig, G. A.; Oehler, R., Lymphocyte Activation Induces Cell Surface Expression of an Immunogenic Vimentin Isoform. Transpl Immunol2012, 27 (2-3), 101-106.
37. Monico, A.; Guzman-Caldentey, J.; Pajares, M. A.; Martin-Santamaria, S.; Perez-Sala, D., Molecular Insight into the Regulation of Vimentin by Cysteine Modifications and Zinc Binding. Antioxidants-Basel 2021, 10 (7).
38. Xu, B.; deWaal, R. M.; Mor-Vaknin, N.; Hibbard, C.; Markovitz, D. M.; Kahn, M. L., The Endothelial Cell-Specific Antibody Pal-E Identifies a Secreted Form of Vimentin in the Blood Vasculature. Mol Cell Biol 2004, 24 (20), 9198-9206.
39. Rose, M. L., Role of Anti-Vimentin Antibodies in Allograft Rejection. Hum Immunol 2013, 74 (11), 1459-1462.
40. von Brandenstein, M.; Puetz, K.; Schlosser, M.; Loser, H.; Kallinowski, J. P.; Godde, D.; Buettner, R.; Storkel, S.; Fries, J. W. U., Vimentin 3, the New Hope, Differentiating Rcc Versus Oncocytoma. Dis Markers 2015, 2015.
41. Mary, S.; Kulkarni, M. J.; Mehendale, S. S.; Joshi, S. R.; Giri, A. P., Differential Accumulation of Vimentin Fragments in Preeclamptic Placenta. Cytoskeleton 2017, 74 (11), 420-425.
42. Huang, L.; Yang, Y. H.; Yang, F.; Liu, S. M.; Zhu, Z. Q.; Lei, Z. L.; Guo, J., Functions of Epcam in Physiological Processes and Diseases (Review). Int J Mol Med 2018, 42 (4), 1771-1785.
43. Pavsic, M.; Guncar, G.; Djinovic-Carugo, K.; Lenarcic, B., Crystal Structure and Its Bearing Towards an Understanding of Key Biological Functions of Epcam. Nature Communications 2014, 5.
44. Martowicz, A.; Spizzo, G.; Gastl, G.; Untergasser, G., Phenotype-Dependent Effects of Epcam Expression on Growth and Invasion of Human Breast Cancer Cell Lines. Bmc Cancer 2012, 12.
45. Schmidt, D. S.; Klingbeil, P.; Schnolzer, M.; Zoller, M., Cd44 Variant Isoforms Associate with Tetraspanins and Epcam. Experimental Cell Research 2004, 297 (2), 329-347.
46. Blazar, M.; Briaire-De Bruijn, I. H.; Rees-Bakker, H. A. M.; Prins, F. A.; Helfrich, W.; de Leij, L.; Riethmuller, G.; Alberti, S.; Warnaar, S. O.; Fleuren, G. J.; Litvinov, S. V., Epidermal Growth Factor-Like Repeats Mediate Lateral and Reciprocal Interactions of Ep-Cam Molecules in Homophilic Adhesions. Mol Cell Biol 2001, 21 (7), 2570-2580.
47. Gutierrez, C.; Schiff, R., Her2: Biology, Detection, and Clinical Implications. Archives of pathology & laboratory medicine 2011, 135 (1), 55-62.
48. Liu, Y.; Vieira, R. M. S.; Mao, L. Simultaneous and Multimodal Antigen-Binding Profiling and Isolation of Rare Cells via Quantitative Ferrohydrodynamic Cell Separation. ACS nano 2022, 17 (1), 94-110.
49. Huang, Y.; Simms, A. E.; Mazur, A.; Wang, S.; León, N. R.; Jones, B.; Aziz, N.; Kelly, T. Fibroblast activation protein-α promotes tumor growth and invasion of breast cancer cells through non-enzymatic functions. Clinical & experimental metastasis 2011, 28, 567-579.
50. Lambert, A. W.; Pattabiraman, D. R.; Weinberg, R. A. Emerging Biological Principles of Metastasis. Cell 2017, 168 (4), 670-691. DOI: 10.1016/j.cell.2016.11.037.
51. Loibl, S.; Gianni, L. HER2-positive breast cancer. The Lancet 2017, 389 (10087), 2415-2429.
52. Cox, K. L.; Devanarayan, V.; Kriauciunas, A.; Manetta, J.; Montrose, C.; Sittampalam, S. Immunoassay methods. Assay Guidance Manual [Internet] 2019.
53. Whipple, R. A.; Balzer, E. M.; Cho, E. H.; Matrone, M. A.; Yoon, J. R.; Martin, S. S. Vimentin filaments support extension of tubulin-based microtentacles in detached breast tumor cells. Cancer Research 2008, 68 (14), 5678-5688. DOI: 10.1158/0008-5472.Can-07-6589.
54. Zhang, H.; Wu, X. S.; Xiao, Y. Z.; Wu, L. Q.; Peng, Y.; Tang, W. M.; Liu, G. N.; Sun, Y.; Wang, J.; Zhu, H. Q.; et al. Coexpression of FOXK1 and vimentin promotes EMT, migration, and invasion in gastric cancer cells. J Mol Med 2019, 97 (2), 163-176. DOI: 10.1007/s00109-018-1720-z.
55. Makise, M.; Nakamura, H.; Kuniyasu, A. The role of vimentin in the tumor marker Nup88-dependent multinucleated phenotype. Bmc Cancer 2018, 18. DOI: ARTN 519 10.1186/s12885-018-4454-y.
56. Kang, C. C.; Yamauchi, K. A.; Vlassakis, J.; Sinkala, E.; Duncombe, T. A.; Herr, A. E. Single cell-resolution western blotting. Nat Protoc 2016, 11 (8), 1508-1530. DOI: 10.1038/nprot.2016.089.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these implementations may be readily apparent to those skilled in the art, and generic principles defined herein may be applied to other implementations. Thus, many changes and modifications may be made to the subject technology, by one having ordinary skill in the art, without departing from the scope of the subject technology. For instance, different numbers of a given module or unit may be employed, a different type or types of a given module or unit may be employed, a given module or unit may be added, or a given module or unit may be omitted.

It should be appreciated that all combinations of the foregoing concepts and additional concepts discussed in greater detail below (provided such concepts are not mutually inconsistent) are contemplated as being part of the subject matter disclosed herein. In particular, all combinations of claimed subject matter appearing at the end of this disclosure are contemplated as being part of the subject matter disclosed herein.

1. An apparatus, comprising:
a droplet generator, comprising:
an oil fluidic line comprising an oil inlet and an oil outlet;
a sample fluidic line comprising a sample inlet and a sample outlet;
a buffer fluidic line comprising a buffer inlet and a buffer outlet;
a droplet generation area where the oil outlet, the sample outlet, and the buffer outlet are coupled; and
an outlet fluidly coupled to the droplet generation area;
a housing comprising a perimeter wall comprising an interior side wall and a lip that define a receptacle,
a microwell assembly positioned within the receptacle and resting on the lip, the microwell assembly comprising a plurality of microwells;
a droplet loading device positioned within the receptacle on top of the microwell assembly, the droplet loading device comprising an inlet manifold assembly comprising an inlet, an outlet manifold assembly comprising an outlet, and a plurality of channels fluidly coupled to the inlet manifold assembly and the outlet manifold assembly, each of the channels have an opening positioned over top of corresponding microwells;
wherein the outlet of the droplet generator is to be fluidly coupled to the inlet of the droplet loading device.

2. The apparatus of claim 1, wherein oil, a sample, and buffer are to be loaded into the corresponding inlets of the droplet loading device, and wherein the droplet generator is to generate droplets that exit the outlet of the droplet generator.

3. The apparatus of claim 2, wherein the droplet loading device is to process the sample without substantial protein loss.

4. The apparatus of claim 2, wherein the oil comprises mineral oil supplemented with Span 80 surfactant.

5. The apparatus of claim 4, wherein a concentration of the Span 80 surfactant is between about 0.2% and about 5% and wherein the buffer comprises a Lysis buffer.

6. (canceled)

7. The apparatus of claim 5, wherein the Lysis buffer comprises sodium dodecyl sulphate (SDS) in water and wherein a concentration of the sodium dodecyl sulphate is between about 0.1% and about 2%.

8. (canceled)

9. The apparatus of claim 1, wherein the droplet generator is used to generate droplets that are substantially stable to enable cell lysis and wherein the droplets being substantially stable comprises the droplets remaining intact without substantial expansion and without substantial shrinkage when exposed to temperatures between about 90° C. and about 100° C. and for incubation periods of between about 1 hour and about 2 hours.

10. (canceled)

11. The apparatus of claim 1, wherein the droplets have a diameter of between about 25 microns and about 95 microns.

12. The apparatus of claim 1, wherein a diameter of the droplets is about 5 microns less than a diameter of the corresponding microwells.

13. The apparatus of claim 1, wherein droplets that exit the outlet of the droplet generator are loaded into the inlet of the inlet manifold assembly to allow the droplets to flow through the channels and be received within corresponding microwells.

14. The apparatus of claim 1, wherein each of the channels has a height of between about 30 microns and about 100 microns.

15. The apparatus of claim 1, wherein the microwells each have a diameter of between about 30 microns and about 100 microns.

16. The apparatus of claim 1, wherein the perimeter wall comprises an end, wherein the housing comprises a lid having a central opening that corresponds to the receptacle, the lid to mate with the end of the perimeter wall.

17. The apparatus of claim 16, wherein the perimeter wall and the lid define corresponding fastener holes, further comprising fasteners that extend through the fastener holes of the perimeter wall and the lid to secure the lid to the perimeter wall.

18. The apparatus of claim 1, further comprising magnets, wherein the perimeter wall comprises an end that defines magnet receptacles and wherein the magnets are to be positioned with the corresponding magnet receptacles.

19. The apparatus of claim 1, further comprising alignment pins, wherein the perimeter wall comprises transverse holes that extend through the perimeter wall and open into the receptacle and wherein the alignment pins are to be positioned within corresponding transverse holes.

20. The apparatus of claim 19, wherein the alignment pins are to adjust the relative position of the droplet loading device to align the openings of the channels of the droplet loading device with the microwells of the microwell assembly.

21. The apparatus of claim 1, further comprising a tube to fluidly couple the outlet of the droplet generator to the inlet of the droplet loading device.

22. The apparatus of claim 1, wherein the microwell assembly comprises a first layer, a second layer, and a third layer, wherein the first layer comprises Polydimethylsiloxane, the second layer comprises gel, and the third layer comprises glass, wherein the gel comprises a polyacrylamide concentration of between about 4% and about 10%, wherein the second layer is positioned between the first layer and the third layer, and wherein the second layer defines the microwells.

23.-26. (canceled)

27. A method, comprising:
loading oil, a sample, and buffer into corresponding inlets of a droplet generator, the droplet generator, comprising: an oil fluidic line comprising the oil inlet and an oil outlet; a sample fluidic line comprising the sample inlet and a sample outlet; a buffer fluidic line comprising the buffer inlet and a buffer outlet; a droplet generation area where the oil outlet, the sample outlet, and the buffer outlet are coupled; and an outlet fluidly coupled to the droplet generation area;
generating droplets using the droplet generator that exit the outlet of the droplet generator;
flowing the droplets to an inlet of an inlet manifold assembly of a droplet loading device positioned within a receptacle of a housing and positioned on top of a microwell assembly comprising a plurality of microwells, the droplet loading device comprising an inlet manifold assembly comprising an inlet, an outlet manifold assembly comprising an outlet, and a plurality of channels fluidly coupled to the inlet manifold assembly and the outlet manifold assembly, each of the channels have an opening positioned over top of corresponding microwells;
directing the droplets to the channels using the inlet manifold assembly; and
flowing the droplets through the channels; and
receiving the droplets in the corresponding microwells.

28-37. (canceled)
